(19) United States
(12) Patent Application Publication
(10) Pub. No.: US 2025/0223641 A1
(43) Pub. Date: Jul. 10, 2025

(54) TARGET ENRICHMENT AND QUANTIFICATION UTILIZING ISOTHERMALLY LINEAR-AMPLIFIED PROBES

(71) Applicant: The Children's Hospital of Philadelphia, Philadelphia, PA (US)

(72) Inventors: Lan LIN, Philadelphia, PA (US); Yi XING, Philadelphia, PA (US); Feng WANG, Philadelphia, PA (US)

(73) Assignee: The Children's Hospital of Philadelphia, Philadelphia, PA (US)

(21) Appl. No.: 18/703,128

(22) PCT Filed: Nov. 9, 2022
(86) PCT No.: PCT/US2022/079537

§ 371 Date: Apr. 19, 2024

Related U.S. Application Data
(60) Provisional application No. 63/277,894, filed on Nov. 10, 2021.

(51) Int. Cl. C12Q 1/6874

(52) U.S. Cl. CPC C12Q 1/6874

(57) ABSTRACT

Transcript Enrichment and Quantification Utilizing Isothermally Linear-Amplified Sequencing (TEQUILA-seq) is a versatile, easy-to-implement, and highly cost-effective method utilizing isothermally linear-amplified capture oligos for targeted sequencing. TEQUILA-seq reduces the per-reaction cost of targeted capture by 2-3 orders of magnitude, as compared to a standard commercial solution. When performed on the Oxford nanopore platform for long-read RNA-seq with multiple gene panels of varying sizes, TEQUILA-seq consistently and substantially enriched transcript coverage while preserving transcript quantification. Profiling of full-length transcript isoforms of 468 actionable cancer genes across 40 breast cancer cell lines representing distinct intrinsic subtypes identified transcript isoforms enriched in specific subtypes and discovered novel transcript isoforms in extensively studied cancer genes such as TP53. Among cancer genes, tumor-suppressor genes were significantly enriched for aberrant transcript isoforms targeted for degradation via mRNA nonsense-mediated decay, revealing a common RNA-associated mechanism for gene inactivation. TEQUILA-seq can be broadly used for targeted sequencing of DNA and RNA in diverse biomedical research settings.

### PRIORITY CLAIM

This application claims benefit of priority to U.S. Provisional Application Ser. No. 63/277,894, filed Nov. 10, 2021, the entire contents of which are hereby incorporated by reference.

### GOVERNMENT RIGHTS

This invention was made with government support under grant numbers GM088342 and GM121827 awarded by the National Institutes of Health. The government has certain rights in the invention.

### INCORPORATION OF SEQUENCE LISTING

The sequence listing that is contained in the file named “CHOP.P0062WO-SequenceListing.xml”, which is 8 KB (as measured in Microsoft Windows®) and was created on Nov. 8, 2022, is filed herewith by electronic submission and is incorporated by reference herein.

### FIELD OF THE INVENTION

The invention is related to methods of making, and methods of using, biotinylated oligonucleotide probes for use in applications such as targeted DNA and RNA sequencing, both long- and short-read, based on a probe capture approach. The methods contemplated herein are both streamlined and cost-effective.

### BACKGROUND OF THE INVENTION

Targeted sequencing approaches, including hybridization-based strategies, are used to enrich next-generation sequencing (NGS) results for sequence regions of interest (ROIs) (Kozarewa et al., 2015). Among its many applications, targeted NGS offers enormous potential as a relatively cost-effective approach for diagnosing Mendelian disease (Sun, Y., et al., 2018). For instance, targeted sequencing using oligonucleotide (oligo) probe hybridization can be used to detect disease-related copy number variants involving one or more exons (Wallace & Bean, 2021). Despite methodological advances, however, commercial biotinylated probes used for targeted sequencing remain expensive, which is an important limitation for targeted sequencing workflows that are already labor-intensive and time-consuming. Thus, there is a need for a highly efficient and cost-effective targeted sequencing technology that can provide the flexibility to interrogate any user-defined gene/sequence panel. Such probe generation and sequence capture technology would be able to detect a wide array of genomic and transcriptomic profiles and changes, including aberrant RNA splicing changes that can cause gene dysregulation and alter cellular phenotypes.

Several approaches for targeted sequencing exist, including hybridization-based strategies, ‘tagmentation’, molecular inversion probes, and single or multiplex PCR amplification (Kozarewa et al., 2015). In the hybridization capture approach, long biotinylated oligo probes are hybridized to sequence ROIs. Sets of sequence ROIs can be sequenced simultaneously by using targeted capture or target enrichment with custom DNA or RNA probes complementary to the sequence ROIs. Commercially available kits for hybridization capture are available from IDT (xGen Lockdown), Agilent (SureSelect), Illumina (TruSeq), Roche (NimbleGen SeqCap EZ), and Life Technologies (Ion TargetSeq) (Kozarewa et al., 2015). Unfortunately, however, currently available commercial capture probes largely rely on predesigned/optimized gene panels that cater to the focus of specific research fields, or use preformulated probe design tools for ad-hoc gene panels of interest. Such custom-designed gene panel probes are usually charged per probe. Thus, a panel containing hundreds of genes would have a prohibitively high initiation cost, as well as a high unit cost per assay.

Targeted sequencing strategies are useful in both DNA and RNA sequencing applications. One focus area of RNA sequencing approach is to study RNA alternative splicing. Alternative splicing of precursor-mRNA is a fundamental gene regulatory process that allows generation of multiple mature mRNA molecules from a single gene, greatly expanding the regulatory complexity and proteome diversity (Nilsen & Graveley, 2010). Over 95% of human multi-exon genes are alternatively spliced (Pan et al., 2008; Wang et al., 2008), resulting in RNA isoforms that can differ in their coding sequences or untranslated regions (UTRs) via basic and complex alternative splicing patterns (Blencowe, 2006; Vaquero-Garcia et al., 2016; Park et al., 2018). These structural differences lead to distinct regulatory properties in mRNA coding capacity, stability, localization, and translation (Baralle & Giudice, 2017). Alternative splicing can be highly cell type-(Shalek et al., 2013; Feng et al., 2021; Joglekar et al., 2021), tissue type-(Ellis et al., 2012), and developmental stage-specific (Xu et al., 2002). Alternative splicing has roles in numerous biological processes, including cell proliferation, survival, homeostasis, migration, and differentiation (Braunschweig et al., 2013; Kalsotra & Cooper, 2011; Paronetto et al., 2016). Splicing aberrations have been implicated in the etiology and progression of human pathologies, including neurological disorders, diabetes, and cancer (Scotti & Swanson, 2016).

Advances in high-throughput sequencing techniques have vastly expanded the inventors' knowledge of gene expression. While enabling accurate identification of individual splice junctions, short-read RNA sequencing (RNA-seq) suffers inherent limitations in unambiguously reconstructing actual transcripts. With typical read lengths of only 100-600 bp, short reads rarely span the entirety of transcripts and, thus, must be computationally assembled, an error-prone process (Steijger et al., 2013). These limitations are particularly pronounced for genes with multiple distantly located alternatively spliced regions (Garber et al., 2011) and for transcripts containing retained introns (Wang & Rio, 2018; Broseus & Ritchie, 2020). By contrast, third-generation sequencing platforms, such as Oxford Nanopore and PacBio, theoretically permit the entire transcript to be sequenced from end-to-end without compromising transcript integrity or requiring computational assembly (Bolisetty et al., 2015; Byrne et al., 2017; Tardaguila et al., 2018; Sahlin et al., 2018; Tang et al., 2020). However, due to the broad dynamic range of isoform expression in the human transcriptome, conventional long-read sequencing techniques with relatively shallow sequencing depth suffer from low sampling sensitivity and sparse coverage of rare transcripts (Stark et al., 2019). As a result, the current barrier of achieving deep isoform sequencing at an affordable cost prevents the widespread adoption of long-read sequencing for complex transcriptome exploration.

Targeted long-read sequencing has emerged as a powerful technique for sequencing genes of interest, offering enormous potential for the detection and quantification of RNA isoforms. Several methods exist for targeted long-read sequencing. Single or multiplex long-range PCR amplification followed by long-read sequencing (Clark et al., 2020) utilizes primer pairs to amplify transcripts of interest from end-to-end. However, such methods can potentially fail to enrich transcripts if their first or last exons are alternatively spliced. Different primers may result in heterogeneous coverage due to amplification bias. Cas9-assisted target enrichment with long-read sequencing (Gabrieli et al., 2018; Gilpatrick et al., 2020), which introduces dual Cas9 cleavage to excise ROIs, can only be used for targeted guide DNA sequencing and achieves less than 5% of on-target reads for enriched regions. Adaptive sampling for real-time selective sequencing on nanopore sequencers (Loose et al., 2016; Payne et al., 2021; Kovaka et al., 2021) ejects uninformative reads selectively while sequencing. However, this method is currently most effective with longer reads (>1350 bp) and has not been optimized for RNA-seq applications with significant number of shorter transcripts less than 1 kb. Probe hybridization-based enrichment is a particularly efficient method (Karamitros & Magiorkinis, 2018). Two RNA Capture-Seq-based (Mercer et al., 2014) approaches, namely RNA Capture Long Seq (Lagarde et al., 2017) and ORF Capture-Seq (Sheynkman et al., 2020), employ tiled oligo probes to enrich cDNAs of interest in conjunction with long-read sequencing.

In summary, despite improvements in targeted sequencing methods, commercially synthesized biotinylated probes are very costly, while accessing and maintaining the human ORFeome library is a time-consuming, costly, and laborious process. Thus, there is a need for an efficient, cost-effective, and user-friendly approach that provides both full-length coverage and sufficient read depth to facilitate comprehensive detection and quantification of full-length transcripts including transcript isoforms resulting from pre-mRNA alternative splicing.

### SUMMARY

Thus, in accordance with the present disclosure, there is provided a method of preparing a panel of biotinylated oligonucleotide probes, the method comprising (a) obtaining a set of oligonucleotides, each comprising a target gene binding sequence at its 5′ end and a primer binding sequence at its 3′ end, wherein each oligonucleotide has the same the primer binding sequence, and wherein the 5′ end of the primer binding sequence comprises a nickase target sequence; (b) incubating the set of oligonucleotides with a primer that hybridizes to the primer binding sequence and with biotinylated dNTP (e.g., biotin-dUTP) under conditions to allow for extension of the primer using the oligonucleotides as a template, thereby producing extended primers complementary to the oligonucleotides, where the extended primers each comprise, from 5′ to 3′, the primer, the nickase target sequence, and a biotinylated probe; (c) nicking the extended primers complementary to the oligonucleotides with a nickase capable of cleaving the extended primers at the nickase target sequence to separate the biotinylated probes and regenerate the primers' 3′ end; (d) extending the regenerated primers 3′ end using the oligonucleotides as templates to displace and release the biotinylated probes; and (e) repeating steps (c) and (d).

In certain embodiments, each oligonucleotide in the set is about 60 to 150 nucleotides long. In certain embodiments, each oligonucleotide in the set comprises a 30 to 120-nucleotide sequence at its 5′ end that is capable of hybridizing to a target gene and a 30-nucleotide primer binding site at its 3′ end. In certain embodiments, the 30-nucleotide primer binding site has one of the following sequences depending on the nickase used and selected from
1) Nt.BspQI: 5′-NGAAGAGCCCTATAGTGAGTCGTATTAGAA-3′;
2) Nt.BstNBI: 5′-NNNNGACTCCCTATAGTGAGTCGTATTAGAA-3′;
3) Nb.AlwI: 5′-NNNNGATCCCCTATAGTGAGTCGTATTAGAA-3′; and
4) Nt.BsmAI: 5′-NGAGACCCTATAGTGAGTCGTATTAGAA-3′,
wherein 5′-CCTATAGTGAGTCGTATTAGAA-3′ is a universal primer sequence and the italicized bases are targeting sequences.

In certain embodiments, within the set of oligonucleotides, the 30 to 120-nucleotide 5′ end sequences are tiled across the sequence of each target gene. In certain embodiments, the oligonucleotides are tiled at about or greater than a density of 0.5×, 1×, or 2× across the sequence of each target gene. In certain embodiments, oligonucleotides are tiled across the targeted gene sequence regions, including, but not limited to genomic DNA or RNA sequences of target genes including the exon sequences, or/and the intronic sequences.

Step (b) may comprise (i) combining the set of oligonucleotides, the primer, deoxynucleotides, and biotinylated dNTP (e.g., biotin-dUTP) and incubating the mixture at 95° C. for 2 min, followed by a slow ramp-down (−0.1° C./s) to 4° C.; and (ii) adding a single-stranded DNA binding protein and a DNA polymerase that exhibits 5′ to 3′ strand displacement activity and incubating at a temperature between 20° C. and 37° C. for initial primer extension. The DNA polymerase that harbors 5′ to 3′ strand displacement activity may include, but is not limited to Klenow Fragment (3′→5′ exo-) DNA polymerase; Hemo KlenTaq DNA polymerase; Bst DNA Polymerase, Large Fragment; Bst DNA Polymerase; Bsu DNA Polymerase, Large Fragment; phi29 DNA Polymerase; and Vent® (exo-) DNA Polymerase.

Steps (c)-(e) may comprise adding a nickase to the reaction and incubating at a temperature between 20° C. and 37° C., such as wherein the incubating occurs for between 30 min and 24 h.

Steps (d) and (e) may occur without any exogenous manipulation.

The method may further comprise (f) isolating and/or purifying the biotinylated probes.

The nickase may be, but is not limited to Nt.BspQI, Nt.BstNBI, Nb.AlwI, or Nt.BsmAI.

The extension of steps (b) and (d) may be performed by a DNA polymerase that harbors 5′ to 3′ strand displacement activity including, but not limited to Klenow Fragment (3′→5′ exo-) DNA polymerase; Hemo KlenTaq DNA polymerase; Bst DNA Polymerase, Large Fragment; Bst DNA Polymerase; Bsu DNA Polymerase, Large Fragment; phi29 DNA Polymerase; and Vent (exo-) DNA Polymerase.

The method may be an isothermal reaction. The method may be performed at a temperature between 20° C. and 37° C.

Also provided is panel of biotinylated oligonucleotide probes made by a method as disclosed herein. Each probe may comprise one or more biotin-NMP residues (e.g., biotin-UMP residues). Each probe may consist of sequences that are complementary to a target nucleic acid sequence, including, but not limited to, a gene's DNA locus, transcript isoforms or an intergenic DNA region.

In yet another embodiment, there is provided method of sequencing a plurality of nucleic acid molecules comprising (a) obtaining a sample comprising the plurality of nucleic acid molecules; (b) hybridizing the panel of probes of any one of claims 18-20 to the plurality of nucleic acid molecules; (c) capturing the hybridized probes using streptavidin beads; (d) amplifying the nucleic acid molecules that were bound to the captured hybridized probes; and (e) sequencing the amplified nucleic acid molecules.

The sequencing may comprise Sanger sequencing, sequencing-by-synthesis, including, but not limited to, Illumina NGS platform sequencing and PacBio long-read sequencing, or nanopore sequencing. The sequencing may comprise long-read sequencing. The sequencing may comprise short-read sequencing.

The streptavidin beads may be magnetic. The sample may be a dsDNA library, including, but not limited to cDNA library and fragmented genomic DNA library, such as wherein the cDNA library was produced by reverse transcription-polymerase chain reaction of an RNA sample. The sequencing may provide a transcriptomic profile, such as wherein the transcriptomic profile includes gene expression changes and RNA splicing changes.

The method may be a method of targeted sequencing of full-length transcripts, non-full-length transcripts or any genomic fragments.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF FIGURES

The following drawings form part of the present specification and are included to further demonstrate certain aspects of the present disclosure. The disclosure may be better understood by reference to one or more of these drawings in combination with the detailed description of specific embodiments presented herein.

FIGS. 1A-B. Schema of TEQUILA-seq. (FIG. 1A) TEQUILA probe synthesis. Oligonucleotides, designed to tile across regions of interest at the desired density, are used as templates to generate biotinylated probes by performing nicking-endonuclease-triggered strand displacement amplification. (FIG. 1B) Poly (A)+ RNA is converted to full-length cDNA using the reverse transcription and template-switching reaction, followed by PCR amplification of cDNA. TEQUILA probes are hybridized to the cDNA library. Targeted cDNA is captured by streptavidin magnetic beads, whereas non-targeted cDNA is washed away. Enriched cDNA is PCR-amplified and subjected to nanopore 1D library construction and sequencing.

FIGS. 2A-D. TEQUILA-seq effectively enriches targeted transcripts. (FIG. 2A) Comparison of target enrichment between the TEQUILA-seq method and the IDT xGen Lockdown Capture-Seq method. Shown are the top 30 genes with the highest number of mapped reads. Bars are colored as blue for “target” genes (including 10 human genes and 3 SIRV genes) or gray for “non-target” genes. Insert: Overall fraction of reads that mapped to “target” genes. Ratio (and error) were calculated as the mean value (and standard deviation) of the percentage of reads that mapped to all target genes in all 3 replicates within the group. (FIG. 2B) Pairwise comparison of Pearson's correlation between replicates based on transcript expression. Pairwise Pearson's correlation coefficients were calculated to measure the similarity between replicates within the same method group and between replicates from different method groups. (FIGS. 2C-D) Comparison of gene expression (FIG. 2C) and number of detected isoforms (FIG. 2D) of target genes between TEQUILA-seq and IDT xGen Lockdown Capture-Seq method. Gene abundance (and error) were calculated as the mean value (and standard deviation) of log 2 (CPM+1) across replicates within the group. Abbreviations: SIRV, spike-in RNA variant.

FIGS. 3A-B. Quantitative comparison of TEQUILA-seq, direct RNA-seq, and 1D cDNA sequencing. (FIG. 3A) Correlation between known spike-in concentration and estimated transcript abundance for 92 spike-in transcripts. (FIG. 3B) Correlation between transcript length and estimated abundance for 15 long SIRVs. Each dot represents the mean value of the measured transcript expression across replicates (n=3 per group) within the group. Error bars of each dot represent the standard deviation of transcript expression across replicates. Dots are colored as blue for “target” genes or gray for “non-target” genes. Regression lines are calculated and drawn for both “target” and “non-target” genes in each method group, respectively.

FIG. 4. Design of oligo pool for TEQUILA probe synthesis. All annotated UTRs and coding sequences of targeted genes are collected as input sequences for designing the oligo pool. Each oligo sequence is 150 nt in length, containing a 30 nt universal 3′-end primer binding sequence (5′-CGAAGAGCCCTATAGTGAGTCGTATTAGAA-3′). The 120 nt 5′-end sequences are designed to achieve the desired tiling density (e.g., 0.5×, 1×, 2×) against the input sequence of targeted genes.

FIG. 5. Pipeline for TEQUILA-seq data analysis. Nanopore 1D sequencing raw reads are base-called using Guppy and aligned to the reference by minimap2. ESPRESSO is used for isoform detection and quantification.

FIGS. 6A-C. Overview of TEQUILA-seq. (FIGS. 6A-B) Schematic of TEQUILA-seq. (FIG. 6A) Single-stranded DNA (ssDNA) oligonucleotides are designed to tile across all annotated exons of target genes and are synthesized using an array-based DNA synthesis technology. Synthesized TEQUILA probes are amplified from ssDNA oligo templates in a single pool using nicking-endonuclease-triggered strand displacement amplification with universal primers and biotin-dUTPs. (FIG. 6B) Full-length cDNAs are synthesized from poly(A)+ RNA by reverse transcription and PCR amplification. TEQUILA probes are then hybridized to cDNAs. Upon capture and washing, cDNA-to-probe hybrids are immobilized to streptavidin magnetic beads, whereas unbound cDNAs are washed away. Captured cDNAs are amplified by PCR and subjected to nanopore 1D library preparation and sequencing. (FIG. 6C) Comparison of TEQUILA-seq vs xGen Lockdown (IDT)-based target enrichment. Main graphs show percentage of reads that map to a given gene (average and standard deviation, n=3 replicates per method), for the 30 genes with the highest number of mapped reads.

FIGS. 7A-C. Sensitive and quantitative transcript detection with TEQUILA-seq. (FIG. 7A) TEQUILA probes were synthesized for 46 External RNA Controls Consortium (ERCC) synthetic transcripts. Detection of transcript isoforms of target genes was compared among standard nanopore 1D cDNA sequencing, direct RNA sequencing, and TEQUILA-seq performed for 4-hours, 8-hours, or 48-hours. Shown are correlations between spike-in concentration and estimated abundance of 92 ERCC spike-in transcripts. (FIG. 7B) TEQUILA probes were synthesized for 5 long spike-in RNA variants (long SIRVs). This probe set was applied to RNAs of human SH-SY5Y neuroblastoma cells spiked-in with 15 long SIRVs. Enrichment towards longer transcripts was compared among the same method groups as in (a). Shown are correlations between transcript length and measured abundance of 15 long-SIRV transcripts. In FIGS. 7A-B, dots and error bars represent average and standard deviation of estimated abundance of individual transcripts (n=3 replicates per method). Hollow dots represent undetected transcripts. For each method group, Pearson's correlation p (FIG. 7A) and regression lines (FIGS. 7A-B) were separately calculated for target and non-target transcripts. Gray area represents the 95% confidence interval of each regression line. (FIG. 7C) TEQUILA probes were synthesized for 221 splicing factor-encoding human genes. TEQUILA-seq of this gene panel was applied to RNAs of SH-SY5Y cells. Preservation of transcript inclusion levels of alternatively spliced exons within target genes was compared among the same method groups as in FIG. 7A, as well as bulk short-read RNA-seq. Shown are correlations between exon-inclusion levels measured using short- and long-read RNA-seq methods for 105 high-confidence exon-skipping events (see Methods) in 221 splicing factor-encoding genes. Each dot represents the exon inclusion level of one exon-skipping event measured from short-vs long-read RNA-seq data (average n=3 replicates per method).

FIGS. 8A-F. TEQUILA-seq analysis of actionable cancer genes in a broad panel of breast cancer cell lines. (FIG. 8A) Summary of gene panel, cell lines, and data processing workflow used for TEQUILA-seq analysis of 468 cancer genes in 40 breast cancer cell lines. (Upper left) TEQUILA probes were synthesized for 468 genes interrogated by MSK-IMPACT (Memorial Sloan Kettering-Integrated Mutational Profiling of Actionable Cancer Targets), an FDA-approved diagnostic test for DNA-based mutation profiling of actionable cancer targets. (Lower left) TEQUILA-seq was performed on 40 cell lines from the ATCC Breast Cancer Cell Panel. These cell lines represent 4 distinct histological subtypes: luminal, HER2 enriched, basal A, and basal B. (Right) Computational workflow for processing TEQUILA-seq data. Raw nanopore data are basecalled and aligned to a reference genome. Next, transcript isoforms are discovered and quantified from long-read alignment data. Finally, aberrant transcript isoforms are detected (see Methods). (FIG. 8B) Enrichment of 468 target genes in MCF7 cell line, based on results from TEQUILA-seq and nanopore 1D cDNA sequencing (non-capture control). Top 2,000 genes with highest measured abundance in each method are shown. (FIG. 8C) UMAP clustering analysis using isoform proportions of all transcript isoforms across 468 genes in 40 cell lines (n=2 per cell line). Each dot represents one replicate of a cell line. (FIG. 8D) Stacked barplot showing proportions of DNMT3B transcript isoforms identified by TEQUILA-seq in 40 cell lines. Red bar: isoform of interest (ENST00000348286); navy bar: canonical isoform (ENST00000328111); lighter blue bars: 3 other most abundant DNMT3B isoforms; gray bars: remaining DNMT3B isoforms. (FIG. 8E) Structures of DNMT3B protein and transcript isoforms. (Upper) Domain annotations for protein isoforms encoded by transcript isoform of interest and canonical transcript isoform of DNMT3B. PWWP, proline-tryptophan-tryptophan-proline domain; ADD, ATRX-DNMT3-DNMT3L-type zinc finger domain; MTase, methyltransferase domain. (Lower) Transcript structures for isoform of interest, canonical isoform, and 3 other most abundant isoforms of DNMT3B. Boxes: exons. Line segments: introns. (FIG. 8F) Violin plots (median, interquartile range) showing distribution of isoform proportions for DNMT3B isoform of interest in different breast cancer histological subtypes. Each dot represents the isoform proportion in a given cell line replicate (n=2 per cell line).

FIGS. 9A-F. Nonsense mediated decay (NMD)-targeted tumor aberrant transcript isoforms are enriched in tumor-suppressor genes. TEQUILA-seq data were used to identify tumor aberrant transcript isoforms, defined as alternative transcript isoforms that are present at significantly elevated proportions in at least one but no more than 4 breast cancer cell lines. (FIG. 9A) Stacked barplot showing number of annotated and novel tumor aberrant isoforms identified across 40 breast cancer cell lines (see Methods). (FIG. 9B) Comparison of tumor aberrant to canonical transcript isoforms of corresponding genes. Pie chart shows distribution of alternative splicing (AS) events associated with identified tumor aberrant isoforms. Number in parenthesis: number of associated tumor aberrant isoforms in each AS event category. (FIG. 9C) Stacked barplots showing abundances (upper panel) and isoform proportions (lower panel) for TP53 transcript isoforms discovered by TEQUILA-seq across 40 breast cancer cell lines. Red bars: isoforms of interest (ESPRESSO: chr17: 1864:802, ESPRESSO: chr17: 1864:391); navy bar: canonical isoform (ENST00000269305); lighter blue bars: 3 other most abundant TP53 isoforms; gray bars: remaining TP53 isoforms. (FIG. 9D) Structures of TP53 transcript isoforms, including isoforms of interest (ESPRESSO: chr17: 1864:802, ESPRESSO: chr17: 1864:391), canonical isoform (ENST00000269305), and the 3 other most abundant TP53 isoforms. Boxes: exons. Line segments: introns. Red octagons: premature termination codons. (FIG. 9E) Stacked barplots showing percentage of 468 cancer genes with NMD-targeted tumor aberrant isoforms. Genes were categorized by their annotations as tumor-suppressor genes (TSGs), oncogenes (OGs) or “Other”. P values: two-sided Fisher's exact test. (FIG. 9F) Box-and-whisker plots (median, interquartile range) with individual data points showing percentage of genes with NMD-targeted tumor aberrant isoforms among all 468 genes detected in a given breast cancer cell line (average n=2 replicates). P values: two-sided paired Wilcoxon test.

FIG. 10. Pairwise comparisons of estimated abundances for transcript isoforms of target genes across TEQUILA-seq and xGen Lockdown-seq libraries. TEQUILA probes and xGen Lockdown probes were generated against a small test panel of 10 brain genes. Both probe sets were applied to the same human brain cDNA sample. Nanopore 1D sequencing data (n=3 experimental replicates per probe set) were generated with comparable sequencing depths. In each pairwise comparison, transcripts of target genes with a CPM>0 in at least one library were included in the plot and used to calculate Pearson's correlation.

FIG. 11. Estimated abundances for transcript isoforms of 10 target brain genes across TEQUILA-seq, xGen Lockdown-seq, and nanopore 1D cDNA sequencing (non-capture control) libraries. Each bar shows the measured abundance of a given gene (average and standard deviation, n=3 experimental replicates per probe set).

FIG. 12. Enrichment of 468 actionable cancer genes in HCC1806, MDA-MB-157, AU-565, and MCF7 breast cancer cell lines, based on results from TEQUILA-seq and nanopore 1D cDNA sequencing (non-capture control). For each cell line, TEQUILA-seq and non-capture control libraries were prepared from the same biological replicate. Each bar shows the percentage of mapped reads derived from all 468 cancer genes.

FIGS. 13A-C. An FGFR2 isoform with a mutually exclusive exon 9 is the predominant splice isoform in basal B breast cancer cell lines. (FIG. 13A) Stacked barplot showing proportions of FGFR2 transcript isoforms identified by TEQUILA-seq in 40 cell lines. Red bar: isoform of interest (ENST00000358487); navy bar: canonical isoform (ENST00000457416); lighter blue bars: 3 other most abundant FGFR2 isoforms; gray bars: remaining FGFR2 isoforms. (FIG. 13B) Structures of FGFR2 protein and transcript isoforms. (Upper) Domain annotations for protein isoforms encoded by transcript isoform of interest and canonical transcript isoform of FGFR2. Immunoglobulin loop domains (Ig-I, Ig-II, and Ig-III), transmembrane domain (TM), and tyrosine kinase domain (TK) are indicated. (Lower) Transcript structures for isoform of interest (ENST00000358487), canonical isoform (ENST00000457416), and 3 other most abundant isoforms of FGFR2. Boxes: exons. Line segments: introns. (FIG. 13C) Violin plots (median, interquartile range) showing distribution of isoform proportions for FGFR2 isoform of interest in different breast cancer histological subtypes. Each dot represents the isoform proportion in a given cell line replicate (n=2 per cell line).

FIGS. 14A-C. An SESN1 isoform with a distal alternative first exon is the predominant splice isoform in basal B breast cancer cell lines. (FIG. 14A) Stacked barplot showing proportions of SESN1 transcript isoforms identified by TEQUILA-seq in 40 cell lines. Red bar: isoform of interest (ENST00000436639); navy bar: annotated protein-coding isoform with the highest average proportion (ENST00000356644, as the reference); lighter blue bars: 3 other most abundant SESN1 isoforms; gray bars: remaining SESN1 isoforms. (FIG. 14B) Structures of SESN1 protein and transcript isoforms. (Upper) Domain annotations for protein isoforms encoded by transcript isoform of interest and reference transcript isoform of SESN1. N-terminal domain (NTD) and C-terminal domain (CTD) are indicated. (Lower) Transcript structures for isoform of interest (ENST00000436639), reference isoform (ENST00000356644), and 3 other most abundant isoforms of SESN1. Boxes: exons. Line segments: introns. (FIG. 14C) Violin plots (median, interquartile range) showing distribution of isoform proportions for SESN1 isoform of interest in different breast cancer histological subtypes. Each dot represents the isoform proportion in a given cell line replicate (n=2 per cell line).

FIG. 15. Identification of tumor-aberrant transcript isoforms across 40 breast cancer cell lines. Stacked barplot shows the number of “cell line-enriched” isoforms, defined as the number of transcript isoforms that had enriched usage in a cell line (see Methods), as a function of the corresponding number of enriched cell lines. “Tumor aberrant” transcript isoforms are cell line-enriched isoforms that showed enriched usage in at least 1 but no more than 4 cell lines (≤10% of all 40 cell lines, solid colors).

FIGS. 16A-B. Confirmation of a splice-site-disrupting mutation causing TP53 splice variants in the HCC1599 cell line. (FIG. 146) RT-PCR validation of splice variants containing exons 6 and 7 of TP53 in the HCC1599 and HCC1806 (control) cell lines. Forward and reverse primers are designed to anneal to exons 6 and 7, respectively. Canonical splicing of exons 6 and 7 corresponds to the 121-bp band. The 689-bp band is a result of intron 6 retention. The 170-bp band is a result of alternative usage of a cryptic 3′-splice site within intron 6. (FIG. 16B) Sanger sequencing identifies a 3′-splice site mutation (A>T) of TP53 intron 6 in HCC1599. Sequencing results are shown for the antisense strands of the TP53 gDNA amplicons from the HCC1599 and HCC1806 (control) cell lines, as well as TP53 cDNA amplicons from the HCC1599 cell line. HCC1806 harbors the wild type 3′-splice site dinucleotide AG, whereas HCC1599 harbors a mutated 3′-splice site dinucleotide TG.

FIGS. 17A-D. A novel aberrant NOTCH1 isoform resulting from a structural deletion is the predominant transcript isoform in the MDA-MB-157 cell line. (FIG. 17A) Stacked barplots showing relative abundances (upper panel) and proportions (lower panel) of NOTCH1 transcript isoforms identified by TEQUILA-seq in 40 cell lines. Red bar: isoform of interest (ESPRESSO: chr9: 9147:301), navy bar: canonical isoform (ENST00000651671); lighter blue bars: 3 other most abundant NOTCH1 isoforms; gray bars: remaining NOTCH1 isoforms. (FIG. 17B) Structures of NOTCH1 transcript isoforms for the isoform of interest (ESPRESSO: chr9: 9147:301), canonical isoform (ENST00000651671), and 3 other most abundant NOTCH1 isoforms. Boxes: exons. Line segments: introns. (FIG. 17C) RT-PCR validation of splice variant with exon junction of exons 1 and 28 of NOTCH1 in MDA-MB-157 and HCC1395 (control) cell lines. Forward and reverse primers are designed to anneal to exons 1 and 28, respectively. The 135-bp band unique to MDA-MB-157 is a result of an intragenic genomic deletion within NOTCH1. (FIG. 17D) Sanger sequencing identifies a ˜41.5 kb genomic deletion in MDA-MB-157. Sequencing results for sense strands of NOTCH1 gDNA amplicons from MDA-MB-157 are shown. Breakpoints of the deletion are located in introns 1 and 27 of NOTCH1.

FIGS. 18A-D. A novel aberrant RBI isoform resulting from a genomic deletion containing exon 22 is the predominant transcript isoform in the HCC1937 cell line. (FIG. 18A) Stacked barplots showing relative abundances (upper panel) and proportions (lower panel) of RBI transcript isoforms identified by TEQUILA-seq in 40 cell lines. Red bar: isoform of interest (ESPRESSO: chr13: 2429:105); navy bar: canonical isoform (ENST00000267163); lighter blue bars: 3 other most abundant RB1 isoforms; gray bars: remaining RB1 isoforms. (FIG. 18B) Structures of RB1 transcript isoforms for the isoform of interest (ESPRESSO: chr13: 2429:105), canonical isoform (ENST00000267163), and 3 other most abundant RB1 isoforms. Boxes: exons. Line segments: introns. (FIG. 18C) RT-PCR validation of splice variants containing exons 21 and 23 of RB1 in HCC1937 and HCC1806 (control) cell lines. Forward and reverse primers are designed to anneal to exons 21 and 23, respectively. Canonical splicing of exons 21 to 23 corresponds to the 283-bp band, with exon 22 inclusion. The 169-bp band unique to HCC1937 is the result of a genomic deletion containing RBI exon 22. (FIG. 18D) Sanger sequencing identifies a 178-bp deletion in HCC1937 containing RB1 exon 22. Sequencing results for antisense strands of RB1 gDNA amplicons from HCC1937 are shown. Breakpoints of the deletion are located in introns 21 and 22 of RB1.

### DETAILED DESCRIPTION

Over the last decade, short-read RNA sequencing (RNA-seq) has been broadly used as the standard approach for transcriptome analysis (Stark et al., 2019). Due to its read length, however, short-read RNA-seq is limited in its ability to resolve full-length transcript isoforms and complex RNA processing events (Park et al., 2018). By contrast, long-read sequencing platforms, such as Pacific Biosciences (PacBio) and Oxford Nanopore Technologies (ONT), can generate reads longer than 10 kb and directly sequence full-length transcript molecules end-to-end (Amarasinghe et al., 2020; Wang et al., 2021). However, a major limitation of long-read sequencing platforms is that their throughput is multiple orders of magnitude lower than that of short-read platforms (Illumina, in particular) (Byrne et al., 2019). This limitation poses a major bottleneck for transcriptome analysis, which requires high sequencing coverage to accurately quantify transcripts and measure isoform proportions, as well as sensitively discover low-abundance transcripts.

Targeted sequencing, which involves enriching specific sequences of interest, provides a useful strategy for substantially enhancing the transcript coverage for a preselected gene panel. To date, several approaches have been developed for targeted long-read RNA-seq. Single or multiplex long-range RT-PCR amplification followed by long-read sequencing utilizes primer pairs placed at terminal exons to amplify target transcripts (Clark et al., 2020). However, this approach may fail to enrich transcripts with novel alternative first or last exons and may not scale up to large gene panels due to issues of primer cross-reactivity and amplification bias. Hybridization capture-based enrichment (Mamanova et al., 2010; Karamitros & Magiorkinis, 2018) using biotinylated capture oligos such as RNA Capture Long Seq (CLS) (Lagarde et al., 2017) is an efficient method for targeted long-read RNA-seq. Nevertheless, commercially synthesized biotinylated capture oligos are costly and can only be used for a limited number of reactions, making the per-sample cost very high for each targeted capture. Sheynkman et al. recently described an alternative hybridization capture-based approach that uses directly synthesized biotinylated capture oligos from open reading frame (ORF) clones (Sheynkman et al., 2020). Still, accessing and operating the human ORFeome library is resource- and time-consuming.

The inventors have developed TEQUILA-seq (Transcript Enrichment and Quantification Utilizing Isothermally Linear-Amplified probes in conjunction with long-read sequencing). A key innovation in TEQUILA-seq is that it uses nicking-endonuclease (nickase)-triggered isothermal strand displacement amplification (SDA) to synthesize large quantities of biotinylated capture oligos from an array-synthesized pool of non-biotinylated oligo templates. This strategy for synthesizing capture oligos makes TEQUILA-seq highly cost-effective and scalable for large gene panels and sample sizes. As such, TEQUILA can be used for generating large pools of capture oligos for any sequence target panel of interest, with substantial cost reduction (at least >200 fold and as high as >10,000 fold) compared to commercially available capture oligos or biotinylated probes. To benchmark its performance, the inventors performed TEQUILA-seq using the ONT platform for multiple gene panels of varying sizes on synthetic RNAs or human mRNAs. To illustrate its biomedical utility, they applied TEQUILA-seq to profile full-length transcript isoforms of 468 actionable cancer genes across a broad panel of 40 breast cancer cell lines representing distinct intrinsic subtypes.

One application of these probes is to be used to hybridize and capture full-length cDNAs for targeted nanopore long-read sequencing. By comparing targeted nanopore long-read sequencing results of a test 10-gene panel and spike-in RNA variants (SIRVs) using TEQUILA probes against widely used commercial probes, the inventors demonstrate that TEQUILA probes achieve significant transcript enrichment, preserve RNA abundance, and effectively detect and measure low-abundance RNA isoforms. Overall, the inventors envision that this highly flexible, efficient, and cost-effective biotinylated probe synthesis method will be of broad utility in various applications in basic and translational research, as well as in clinical diagnostics.

The TEQUILA probes envisioned according to the invention are preferable and superior to other available probes in that they are specific and do not include foreign adaptor sequences in their final format. Nickases, e.g., Nt.BspQI, Nt.BstNBI, Nb.AlwI, and Nt.BsmAI, bind to their recognition sequences within the double-stranded DNA substrate. After binding, nickases hydrolyze only one strand of DNA to produce site-specific nicks, which can serve as initiation sites for linear strand displacement amplification. According to the proprietary TEQUILA probe synthesis methods described herein, the recognition sequence of Nt.BspQI is designed within the universal adaptor region. The nickase can cleave out the universal adaptor sequences from the newly synthesized strand, so that the resulting TEQUILA probes are free of any additional sequences other than complementary sequences against the targeted sequences of interest.

Furthermore, the proprietary methods of the invention reduce the occurrence of PCR amplification-related probe synthesis errors. According to the methods of the invention (i.e., the method for TEQUILA probe synthesis), as the Klenow Fragment (3′→5′ exo-) DNA polymerase extends the upstream strand, the downstream strand is displaced into a single-stranded form, while the nicking site is regenerated by Nt.BspQI. The continuous repetitive actions of nickase and DNA polymerase result in linear amplification of one strand of the DNA molecule. Newly synthesized TEQUILA probes are always generated from the original oligo templates, which largely reduces the possibility of accumulating amplification errors. By contrast, in PCR-based methods, probes are synthesized using templates generated in previous cycles, such that synthetic errors can be exponentially amplified.

An additional advantageous feature of the proprietary TEQUILA probes described herein is that they contain multiple biotinylated-U residues. By contrast, current and commercially available probes are labeled with a single 5′-biotin moiety.

Another advantage of the invention is that the proprietary TEQUILA probes can still be used for hybridization and capture even when the oligos are truncated. In prior art and currently available 5′ biotinylated probe synthesis, oligos are synthesized by adding one base at a time using chemical reactions. Some truncated oligos are inevitably generated, and the 5′ biotin modification can be lost. Loss of 5′ biotin can also happen when the probes are sheared or degraded during long-time storage. In either case, although these probes can hybridize to the targeted sequences, probes without the 5′ biotin modification cannot be captured by streptavidin beads, and the capture efficiency is impaired. By contrast, the proprietary TEQUILA probes incorporate multiple biotinylated-UMPs. As a result, truncated oligos can still be used as probes for hybridization and capture.

An additional advantage of the TEQUILA probes is that the isothermal reaction eliminates the need for a thermal cycler. TEQUILA probe synthesis is an isothermal reaction, which only requires a mild condition (room temperature to 37° C.) for the enzymes. It can be easily set up to generate probes at scale.

Furthermore, the methods described herein are highly cost-effective. The cost of synthesizing TEQUILA probes is significantly reduced (by at least 2 orders of magnitude) compared to current commercial methods. For example, the cost of purchasing a custom-defined set of biotinylated probes (IDT) for a 200-gene panel is $9,000 for a total of 16 reactions, at ˜$562 per capture reaction. In contrast, a Twist oligo pool for the same 200-gene panel is $1,820. This can be used to generate TEQUILA probes for over 10,000 reactions, at ˜$0.2 per reaction, or ˜$0.4 per reaction when factoring in the cost of consumables and enzymes used for probe synthesis.

An additional advantageous feature of the invention is the potential to scale-up biotinylated probe production. Though not wishing to be bound by the following theory, the reaction yield of biotinylated oligos depends, at least in part, on the incubation time, dNTP concentration, and half-life of enzyme activity. What the inventors have observed in previous results is that the probe yield increased with longer incubation time (4 vs. 12 h), indicating the potential for scale-up during biotinylated probe production.

### II. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Protocol for TEQUILA Probe Synthesis

Protocols and methods for producing TEQUILA probes are provided below. As described in this application, the proprietary methods yield novel synthetic capture probes. The probes are unique and cost-effective. In conjunction with long-read RNA-seq, they enable full-length coverage and sufficient read depth, facilitating comprehensive detection and quantification of full-length transcripts including transcript isoforms resulting from pre-mRNA alternative splicing.

### Reagents

Reverse complimentary oligo: 5′-TTCTAATACGACTCACTATAGGGCTCTTCG-3′ (standard desalting).
Biotin-16-aminoallyl-2′-dUTP (Trilink, N-5001) or other type of biotinylated dNTP that can incorporate into new synthesized DNA strand during amplification by DNA polymerase (such as Biotin-11-dUTP)
Deoxynucleotide (dNTP) Solution Set0.1M Dithiothreitol (DTT)
T4 Gene 32 Protein (NEB, M0300S) or other single-stranded DNA binding protein
Klenow Fragment (3′→5′ exo-) DNA polymerase
Nt.BspQI (NEB, R0644S) or other type of nicking endonuclease that cleaves only one strand of DNA on a double-stranded DNA substrate.
10× buffer (1M NaCl, 500 mM Tris-HCl, 100 mM MgCl₂)
Ethanol (absolute)
RNase-/DNase-free water
Agencourt AMPure XP (Beckman, A63881)

### Equipment and Consumables

Nuclease-free PCR tubes, 0.2 ml (Eppendorf, cat. no. 951010006)·
DNA LoBind tubes, 1.5 ml (Eppendorf, cat. no. 022431021)
Benchtop centrifuges or microcentrifuges for 1.5-ml and 0.2-ml tubes
PCR thermocycler(s) suitable for 0.2-ml tubes, 0.3-ml 96-well plates
Pipettors, 1-10 μl, 20 μl, 200 μl, 1,000 μl
Vortex mixer
Bioanalyzer or Tapestation (Agilent Technologies)
NanoDrop spectrophotometer or Qubit fluorometer (Thermo Scientific)

Oligo pool design and synthesis. The inventors' method can be applied to any sequence set that a user wishes to target. In their current application of TEQUILA probes, the inventors aim to resolve complex alternative splicing of genes of interest. Thus, all annotated UTRs and coding sequences of targeted genes are collected as input sequences for designing the oligo pool. Each oligo sequence is 150 nt in length, containing a 30 nt universal 3′-end primer binding sequence (5′-CGAAGAGCCCTATAGTGAGTCGTATTAGAA-3′). The 120 nt 5′-end sequences are designed to achieve the desired tiling density (e.g., 0.5×, 1×, 2×) against the input sequence of targeted genes (FIG. 4).

The designed oligo pool is synthesized by silicon-based DNA Synthesis platform (such as Twist Bioscience). Synthesized oligos are resuspended in TE buffer (10 mM Tris, 0.1 mM EDTA, pH 8.0) and diluted to 2-5 ng/ul. Oligos stored at −20° C. are stable for at least 24 months.

### Nickase-Induced Strand Displacement Amplification

1. Combine the following components in a PCR tube:

| | Volume | Final | |
|---|---|---|---|
| Component | (μl) | concentration | |
| Oligo pool (2 ng/μl) | 5 | 0.2 | ng/μl |
| RC_oligo (5 μM) | 2.5 | 0.25 | μM |
| 10X Buffer | 5 | 1× | |
| DTT (100 mM) | 1 | 2 | mM |
| dATPs/dCTPs/dGTPs (30 mM) | 1 | 0.6 | mM |
| dTTPs (20 mM) | 1 | 0.4 | mM |
| Biotin-16-aminoallyl-2′-dUTP (5 mM) | 2 | 0.2 | mM |

| | | |
|---|---|---|
| Nuclease-free water | 21.5 | — |
| Total Volume | (39) | — |

2. Mix and briefly centrifuge solution.
3. Heat mixture at 95° C. for 2 min, followed by a slow ramp-down (−0.1° C./s) to 4° C.
4. Add the following components to the reaction:

| Component | Volume (μl) | Final concentration | |
|---|---|---|---|
| T4 Gene 32 Protein (~300 μM) | 1 | ~5-6 | μM |
| Klenow Fragment (3′→5′ exo-) | 8 | 0.8 | U/μl |

| | | |
|---|---|---|
| DNA polymerase (5 U/μl) | | |
| Total Volume | (48) | — |

5. Incubate at 37° C. for 2 min for initial primer extension.
6. Add nickase to the reaction:

| Component | Volume (μl) | Final concentration |
|---|---|---|
| Nt.BspQI (10 U/μl) | 2 | 0.4 U/μl |
| Total Volume | (50) | — |

7. Incubate at 37° C. for 30 min to 16 h, 80° C. for 20 min, 4° C. hold.
8. Prepare the AMPure XP beads for use; resuspend by vortexing.
9. Transfer 50 μl of reaction products to a clean 1.5 ml Eppendorf DNA LoBind tube.
10. Add 90 μl (1.8×) of resuspended AMPure XP beads and mix by pipetting.
11. Incubate on a Hula mixer (rotator mixer) for 5 min at room temperature.
12. Prepare 2 ml of fresh 80% ethanol in nuclease-free water.
13. Spin down sample and pellet on a magnet. With tube on magnet, pipette off supernatant.
14. Keeping tube on magnet, wash beads with 1 ml of freshly prepared 80% ethanol without disturbing pellet.
15. Remove 80% ethanol using a pipette and discard.
16. Repeat steps 14-15.
17. Spin down and place tubes back on magnet. Pipette off any residual ethanol. Allow to air dry for ˜30 s, being careful not to dry pellet to the point of cracking.
18. Remove tubes from magnetic rack and resuspend pellet in 51 μl of nuclease-free water. Incubate for 5 min at room temperature.
19. Pellet beads on a magnet until eluate is clear and colorless.
20. Remove and retain 50 μl of elute into a clean 1.5 ml Eppendorf DNA LoBind tube.
21. Measure concentration by Nanodrop spectrophotometer.

### Example 2—Results

Targeted RNA sequencing based on the probe capture approach has the potential to advance detection of transcript complexity and abundance for a desired set of genes. However, the cost of commercially available probes remains prohibitively high, preventing application of the method to studies where a large number of samples need to be processed. Towards this end, the inventors developed TEQUILA, a cost-effective probe synthesis strategy that can be coupled to any targeted high-throughput sequencing approaches, including both long- and short-read sequencing on either DNA or RNA targets. In this disclosure, the inventors demonstrate one such application, targeted nanopore long-read sequencing, which showcases the utility of such technology in terms of capture efficiency, dynamic range, sensitivity, and accuracy. The goal of applying TEQUILA in targeted long-read RNA sequencing is to enhance full-length isoform detection and quantitation for a select set of genes in a single assay at desired sequencing depth.

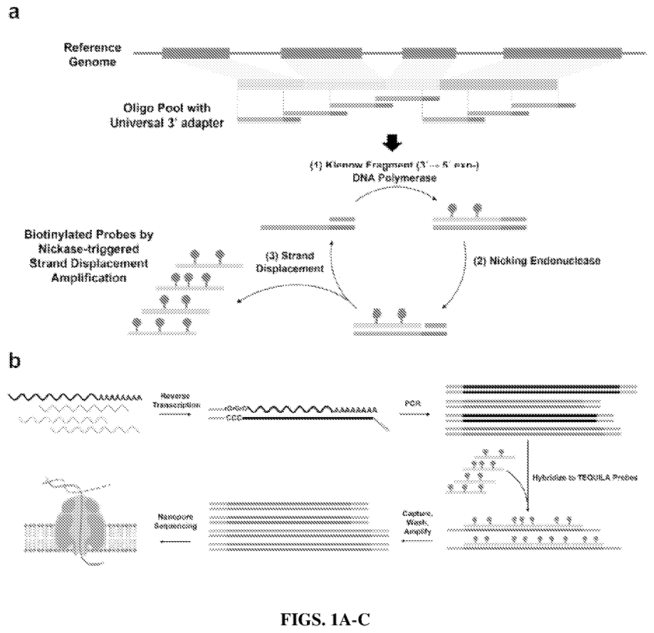

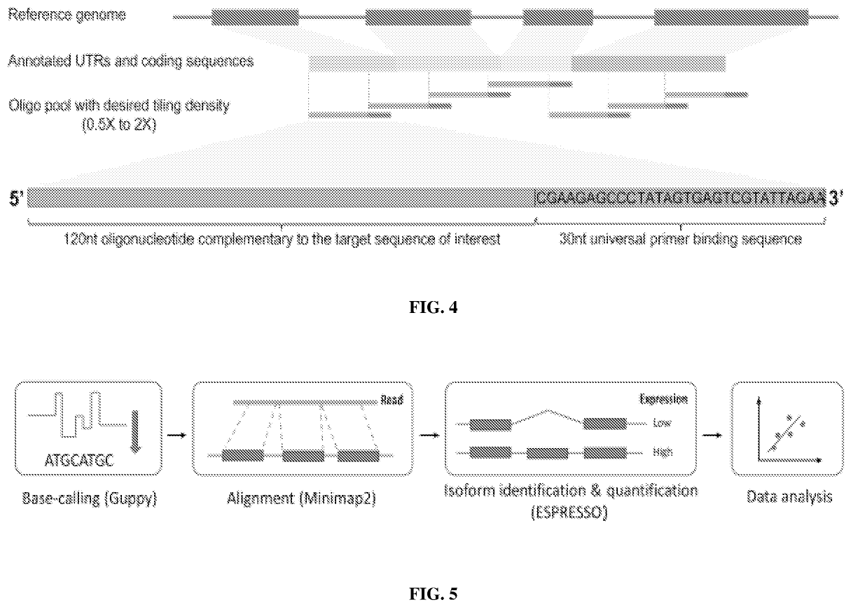

TEQUILA-seq workflow. The TEQUILA-seq platform applies biotinylated TEQUILA probes (synthesized using the proprietary TEQUILA synthesis method described herein) to capture cDNA sequences for targeted long-read sequencing. Specifically, to synthesize TEQUILA probes, a pool of oligos is designed to tile across annotated exon sequences for genes of interest. Next, nickase-triggered strand displacement amplification is performed on the pooled oligos using universal primers in the presence of biotin-dUTPs (FIG. 1A). The TEQUILA-seq workflow is composed of the following steps (FIG. 1B). The full-length cDNA library from poly(A)+RNA is prepared by reverse transcription and PCR pre-amplification. The purified TEQUILA probes are hybridized to the cDNA library. The targeted-cDNA: probe hybrid is immobilized to streptavidin magnetic beads, whereas non-targeted cDNA is washed away. Enriched cDNA is further PCR-amplified and subjected to nanopore 1D library construction and sequencing. Resulting raw reads are base-called using Guppy and aligned to the reference by minimap (Sun et al., 2018). Finally, a bioinformatics program, ESPRESSO (manuscript in preparation), is used for isoform detection and quantification (FIG. 5).

TEQUILA-seq effectively enriches targeted transcripts. To evaluate the performance of TEQUILA-seq, the inventors designed a gene test panel composed of 10 brain-expressed genes, HTT, MAPT, RBfox1, NRXN1, NUMB, DAB1, Grin1, Scn8a, PSD95, and ApoER2. These genes were selected based on their reported long transcript length, complex alternative splicing pattern, or specific RNA isoforms indicative of physiological or pathological conditions in human brain. The inventors intend to use this panel to test the ability of TEQUILA-seq to capture transcripts with extremely long length. The longest annotated isoform for each of these 10 genes ranges from 3,647 to 13,481 nt. Among the 10 genes, 8 genes have 3′UTR sequences >2,500 nt, with the longest up to 5,435 nt.

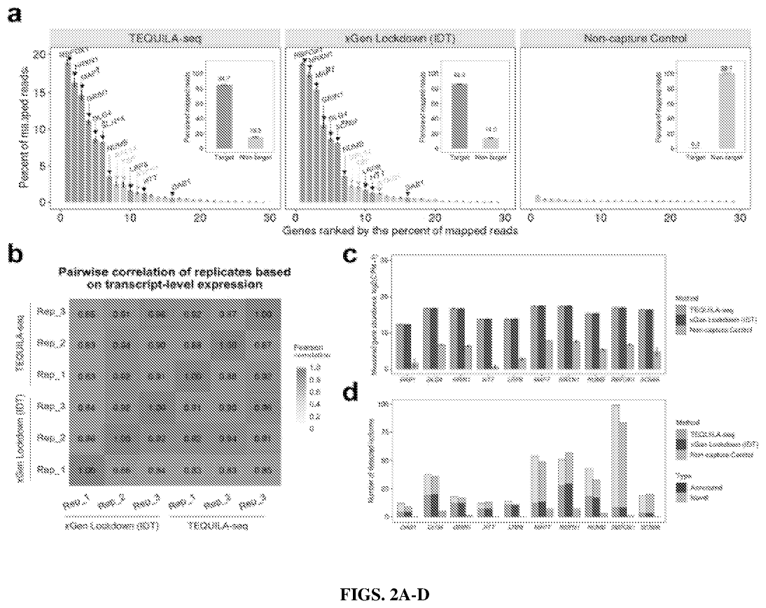

To benchmark, the inventors compared performances of TEQUILA-seq and a commercial standard, xGen Lockdown probe-based capture sequencing (IDT) (FIG. 2A). They applied both methods on the same human brain total RNA sample pooled from multiple donors. Both TEQUILA-seq probes and xGen Lockdown probes were designed with 1× tiling density against the 10 genes. Standard whole-transcriptome 1D cDNA sequencing without capture enrichment was performed as control (Non-capture Control). Three technical replicates generated for each of the 3 methods resulted in comparable numbers of raw nanopore sequencing reads.

The findings showed that TEQUILA-seq has comparable performance to xGEN Lockdown Capture-Seq in enriching targeted transcripts. Both methods produced an on-target rate of ˜85%, with similar fold enrichment (˜280× fold). In terms of capture specificity, all 10 genes of interest were highly enriched in both methods, and their ranks by detected abundance were largely consistent (FIG. 2A). To evaluate reproducibility, the inventors performed pairwise comparisons by calculating the degree of similarity in transcript expression across 3 replicates of each method. Technical replicates from TEQUILA-seq and xGEN Lockdown Capture-Seq were statistically indistinguishable (FIG. 2B). Compared to the non-capture control group in which some genes of interest were merely detected due to insufficient depth, both TEQUILA-seq and xGen Lockdown Capture-Seq were able to enrich all 10 genes and achieved a similar fold enrichment for each individual gene at both the gene and isoform levels (FIGS. 2C-D).

Overall, the inventors demonstrated that TEQUILA-seq provided comparable capture efficiency, specificity, and reproducibility compared to a widely used commercial method.

Transcript characterization and quantification. The inventors systematically evaluated the ability of TEQUILA-seq to characterize and quantify transcripts by employing synthetic spike-in RNA variant (SIRV) set-4 (SIRV-set4, Lexogen). Two groups of artificial genes in SIRV-set4 were used to assess different aspects of sequencing performance: 1) External RNA Controls Consortium (ERCC) mix, composed of 92 non-isoform ERCC transcripts of unique sequence identity at concentrations ranging 6 orders of magnitude, was used to assess the accuracy of quantification; and 2) long SIRVs, comprising 15 transcripts with sizes ranging 4,000-12,000 nt, was used to assess size coverage of the method.

TEQUILA-seq probes were synthesized for 46 transcripts in 2 subgroups of the ERCC module, and 5 transcripts covering all designed sizes from the long-SIRV module. Remaining transcripts without probes served as non-target controls. A total of 5 μg of SIRV-set4 RNAs was spiked into 200 ng of total RNA isolated from the SH-5YSY neuroblastoma cell line. For comparison, the inventors performed whole-transcriptome 1D cDNA-seq and TEQUILA-seq using the above mixture of RNAs with 3 replicates per method. The also generated 3 replicates of direct RNA-seq data from a mixture of 500 ng SH-5YSY poly(A)+RNA and 5 ng of SIRV-set4 RNA. To assess the relationship between sequencing depth and capture quantification of TEQUILA-seq, the inventors also generated a series of TEQUILA-seq data with sequencing times of 4, 8, and 48 h.

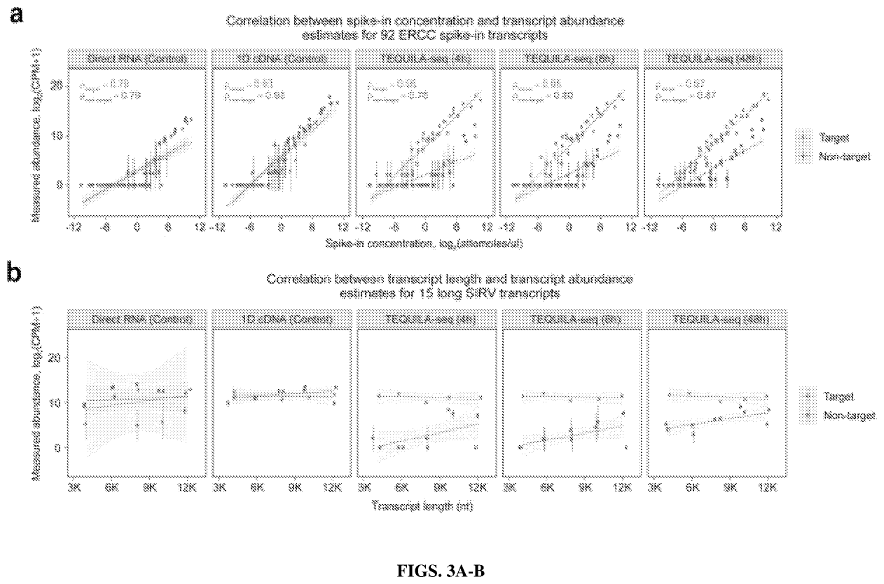

To assess the quantitative accuracy for gene abundance, the inventors compared the ERCC transcript quantification among TEQUILA-seq, direct RNA-seq and 1D cDNA-seq (FIGS. 3A-B). TEQUILA-seq enriched targeted ERCC transcripts with concentrations as low as 0.0625 attomoles/μl. By comparison, in the direct RNA-seq and 1D cDNA-seq controls, the lowest concentration for ERCC transcript that the inventors could consistently detect across replicates was ˜10 attomoles/μl. In addition, TEQUILA-seq retained linear quantification of ERCC standard abundance and provided a more accurate measurement for targeted ERCC transcripts (Pearson's r≥0.95) than direct RNA-seq (Pearson's r=0.79) or 1D cDNA-seq (Pearson's r=0.93) (FIG. 3A). Measurement of ERCC transcripts not targeted by TEQUILA-seq was less accurate (Pearson's r=0.76-0.87) than the measurement in 1D cDNA-seq (Pearson's r=0.93), consistent with the nature of the carry-over of non-specific transcripts. Detection of targeted ERCC transcripts by TEQUILA-seq slightly improved with longer sequencing time (FIG. 3A). The 48-h TEQUILA-seq run generated an average of 10M raw reads, which was 6- to 8-folds compared to data generated for the 4-h (average 1.2M reads) and 8-h (average 1.6M reads) sequencing runs. However, measurement accuracy did not increase significantly with increased run time (Pearson's r=0.95 in 4- or 8-h TEQUILA-seq vs Pearson's r=0.97 in 48-h TEQUILA-seq). This finding indicates that TEQUILA-seq with relatively shallow overall sequencing depth preserves quantification for transcript abundance.

To assess the ability of TEQUILA-seq to maintain measurement accuracy for long transcripts, the inventors compared the correlation between transcript length and detected abundance by analyzing the long SIRV module. The equal abundance of the targeted long SIRV transcripts at each designed length was well preserved in the TEQUILA-seq data (FIG. 3B).

### Example 3—Materials and Methods

Cell lines. The SH-SY5Y human neuroblastoma-derived cell line (ATCC, #CRL-2266) was cultured in DMEM/F-12 (Gibco, #11330032) supplemented with 10% fetal bovine serum (FBS, Corning, #45000-734) and 100 U/ml penicillin-streptomycin (Gibco, #15140122). SH-SY5Y cultures were maintained at 37° C. in a humidified chamber with 5% CO₂. The cell line was authenticated by short tandem repeat analysis and examined to be mycoplasma-free.

RNA extraction and preparation. Synthetic SIRVs (Lexogen, #025.03 and #141.01) were aliquoted immediately upon arrival (5 ng per tube). One aliquot was further diluted by 1:1000 to 5 pg/μl. RNA purity and individual concentrations of SIRVs were verified by the manufacturer. Normal human brain total RNA (50 μg; Clontech Cat. #636530, Lot. #2006022) was isolated from pooled tissues of multiple donors as indicated by the manufacturer. Total RNA from the SH-SY5Y cell line was extracted with Trizol reagent (Invitrogen, #15596018). RNA concentrations and RNA integrity were measured by NanoDrop 2000 Spectrophotometer and Agilent 4200 TapeStation, respectively.

Direct RNA library construction and nanopore sequencing. A total of 20 μg of total RNA was subjected to poly(A)+ RNA selection using Dynabeads mRNA DIRECT purification kit (Invitrogen, #61011) following the manufacturer's instructions. Approximately 500 ng of the resulting poly(A)+ RNA, along with 5 ng of SIRVs, were pooled in one tube as input for direct RNA library generation. Libraries were made by following the standard SQK-RNA002 protocol with the optional reverse transcription step included. All libraries were loaded onto R9.4.1 flow cells and sequenced on MinION/GridION devices (Oxford Nanopore Technologies).

cDNA synthesis. A total of 200 ng of total RNA along with 5 μg of SIRVs was used as the template for cDNA synthesis by following the SMART-seq2 protocol with some modifications. The reverse transcription and template-switching reaction was performed by Maxima H minus reverse transcriptase (Thermo Scientific, #EP0751) under the following conditions: 42° C. for 90 min, 85° C. for 5 min. PCR amplification of first-strand cDNA using KAPA HiFi ReadyMix (KAPA Biosystems, #KK2602) was performed by incubating at 95° C. for 3 min, followed by 11 cycles of (98° C. for 20 s, 67° C. for 20 s, 72° C. for 5 min) with a final extension at 72° C. for 8 min. PCR products were purified using 0.8× volume of SPRIselect beads (Beckman Coulter, #B23318). Amplified cDNA was measured by Qubit dsDNA HS assay and Agilent HS D5000 ScreenTape assay on 4200 TapeStation.

1D library construction and nanopore sequencing. 1D nanopore libraries were constructed using 1 μg of amplified cDNA according to the standard SQK-LSK109 protocol. Briefly, cDNA products were end-repaired and dA-tailed using NEBNext Ultra II End Repair/dA-Tailing Module (NEB, #E7546) by incubating at 20° C. for 20 min and 65° C. for 20 min. End-prepared cDNA was purified with 1× volume of AMPure XP beads and eluted in 60 μl of nuclease-free water. Adapter ligation was performed by using NEBNext Quick T4 DNA ligase (NEB, #E6056) at room temperature for 10 min. After ligation, libraries were purified with 0.45× volumes of AMPure XP beads and short fragment buffer to enrich all fragments equally. Final libraries were loaded onto R9.4.1 flow cells and sequenced on MinION/GridION devices (Oxford Nanopore Technologies) for the desired time.

IDT capture probe synthesis. IDT Lockdown probes were designed and synthesized using the Integrated DNA Technologies (IDT) oligo synthesis service. The probes are 120 nt 5′-end biotinylated oligos with 1× tiling density that tile all annotated UTR and coding sequences of targeted genes.

Hybridization and capture. All steps for hybridization and capture experiments were adopted from the ORF Capture-Seq protocol and the protocol of “Hybridization capture of DNA libraries using xGen Lockdown probes and reagents” from IDT. Briefly, ˜500 ng of amplified cDNA was denatured at 95° C. for 10 min and then incubated with either 3 pmol of xGen Lockdown probes (IDT) or 100 ng of TEQUILA probes at 65° C. for 4-12 h. Next, 50 μl of M-270 streptavidin beads (Invitrogen) were added and incubated at 65° C. for 45 min, immediately followed by a series of high-temperature and room temperature washes, according to the IDT xGen Lockdown protocol. The beads were resuspended in 40 μl of TE buffer.

Post-capture amplification and nanopore sequencing. On-bead PCR was performed using the KAPA HiFi ReadyMix by incubating at 95° C. for 3 min, followed by 12 cycles of (98° C. for 20 s, 67° C. for 20 s, 72° C. for 5 min) with a final extension at 72° C. for 8 min. PCR products were purified using 0.75× volumes of SPRIselect beads. Amplified cDNA was subjected to 1D library construction and sequencing, as described above.

Preprocessing of nanopore sequencing data. Guppy (v4.0.15) from Oxford Nanopore Technologies was used for base-calling direct RNA and cDNA data. Reads were aligned to the hg19 reference genome with GENCODE v34 annotations using minimap2 (v2.17) with parameters “-a -x splice -ub -k 14 -w 4-secondary=no-junc-bed”. Reads corresponding to SIRVs were aligned against the SIRV genome from Lexogen (SIRV-set1/SIRV-set4) using minimap2 with the same parameters.

Detection and quantification of isoforms. Full-length isoforms were detected and quantified from raw read alignment data using ESPRESSO (v1.2.2) (manuscript in preparation), a bioinformatics program that can effectively improve splice junction accuracy and isoform quantification. Transcripts with an average of at least 3 mapped reads across all replicates of a sample group were kept for downstream analysis.

Performance comparison between TEQUILA-seq and IDT xGen Lockdown Capture-Seq. Three methods, ‘TEQUILA-seq capture’, ‘xGen Lockdown (IDT) capture’ and ‘No capture control’ were used to obtain nanopore long-read sequencing results from pooled human brain RNA. Each group has 3 technical replicates. All replicates were sequenced, aligned, and quantified separately. The inventors calculated pairwise Pearson's correlations based on transcript expression from target genes to measure the reproducibility within each group and the similarity between groups. For each replicate in a group, the inventors calculated the on-target ratio as the number of reads that mapped to target genes in the sam/bam file, divided by the total number of reads that aligned to the human genome and SIRV genome. Next, the mean value and standard deviation based on the on-target ratios of each replicate within a group were calculated to represent the overall on-target ratio for that group. In the detection of annotated and novel isoforms of 10 target genes, to decrease the false positive rate, the inventors set a more stringent filter that only considers transcripts with at least 3 mapped reads in all replicates (n=3) in at least one of the ‘TEQUILA-seq’ and ‘xGen Lockdown (IDT)’ groups.

Evaluation of TEQUILA-seq using SIRV-set4 kit. Three methods, ‘TEQUILA-seq capture’, ‘1D cDNA control’ and ‘Direct RNA control’, were used to obtain nanopore long read sequencing results from the SH-5YSY RNA spiked in with SIRV-set4. Each group has 3 technical replicates. All replicates were sequenced, aligned, and quantified separately. To evaluate the maintenance of gene abundance, the inventors used the ERCC panel and calculated the Pearson correlation between the spike-in concentration and the transcript abundance estimate for 46 target genes and 46 non-target genes, respectively. To check whether ‘TEQUILA-seq’ has a potential bias to longer transcripts, the inventors calculated the Pearson correlation between transcript length and estimated abundance for 5 targeted long SIRVs and 10 non-targeted long SIRVs, respectively.

### Example 4—Results

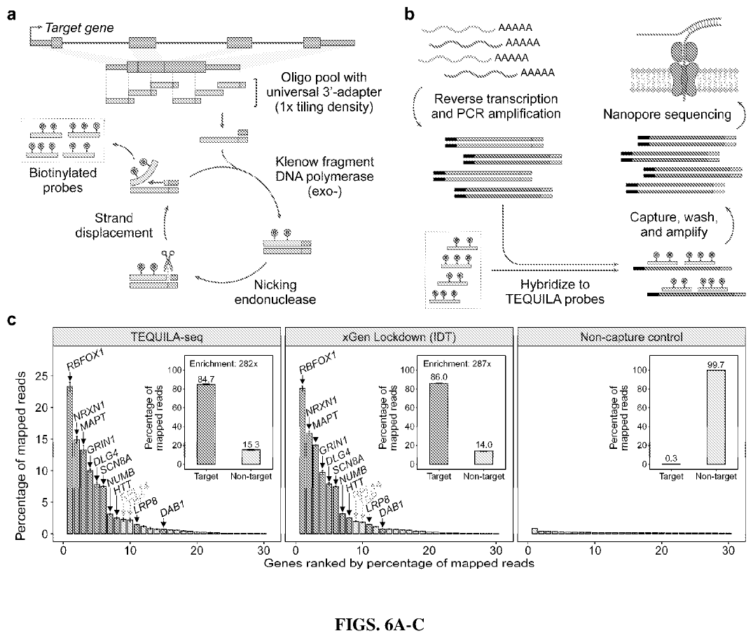

Overview of TEQUILA-seq. The inventors developed TEQUILA as a versatile, easy-to-implement, and highly cost-effective approach for generating large quantities of biotinylated capture oligos for any gene panel (FIG. 6A). First, single-stranded DNA (ssDNA) oligos are designed to tile across all annotated exons of target genes and are synthesized using an array-based DNA synthesis technology. Next, TEQUILA probes are amplified from ssDNA oligo templates in a single pool using nickase-triggered SDA with universal primers and biotin-dUTPs. SDA enables isothermal amplification of internally biotinylated oligos through repeated cycles of nicking and extension reactions using a strand displacement DNA polymerase and pre-designed nickase-targeted nicking sites. This process allows large quantities of capture oligos to be generated from starting templates. The resulting pool of TEQUILA probes can be used to capture full-length cDNA molecules of genes of interest. Because of the low-cost ssDNA oligo pool and the large probe synthesis output, TEQUILA substantially reduces the setup and per-reaction costs of targeted capture compared to commercial methods (Supplementary Tables 1 and 2). For example, a custom set of xGen biotinylated oligos from Integrated DNA Technologies (IDT) for a 6,000-probe panel is $13,000 for 16 reactions (˜$813/reaction). By contrast, the setup cost of TEQUILA probe synthesis for the same 6,000-probe panel is $1,820, and this pool can be used to synthesize TEQUILA probes for >10,000 reactions, at ˜$0.43/reaction when considering the costs of reagents and consumables.

When coupled with long-read RNA-seq, TEQUILA-seq is designed to provide high coverage of full-length transcripts to facilitate comprehensive discovery and accurate quantification of transcript isoforms (FIG. 6B). Briefly, full-length cDNAs are synthesized from poly(A)+ RNAs by reverse transcription and PCR amplification. TEQUILA probes are then hybridized to cDNAs. Upon capture and washing, cDNA-to-probe hybrids are immobilized to streptavidin magnetic beads, whereas unbound cDNAs are washed away. Captured cDNAs are further amplified by PCR and subjected to nanopore 1D library preparation and sequencing. Finally, TEQUILA-seq data are analyzed by the inventors' ESPRESSO software, designed for robust transcript analysis using error-prone long-read RNA-seq data.

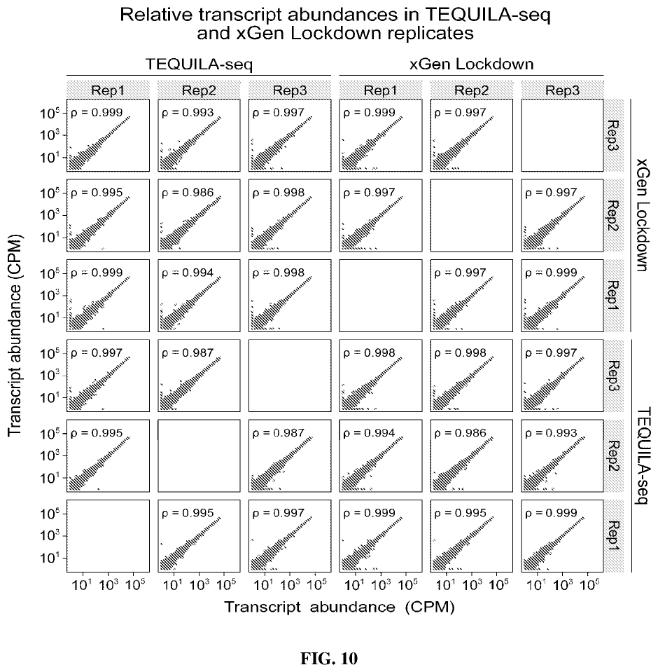

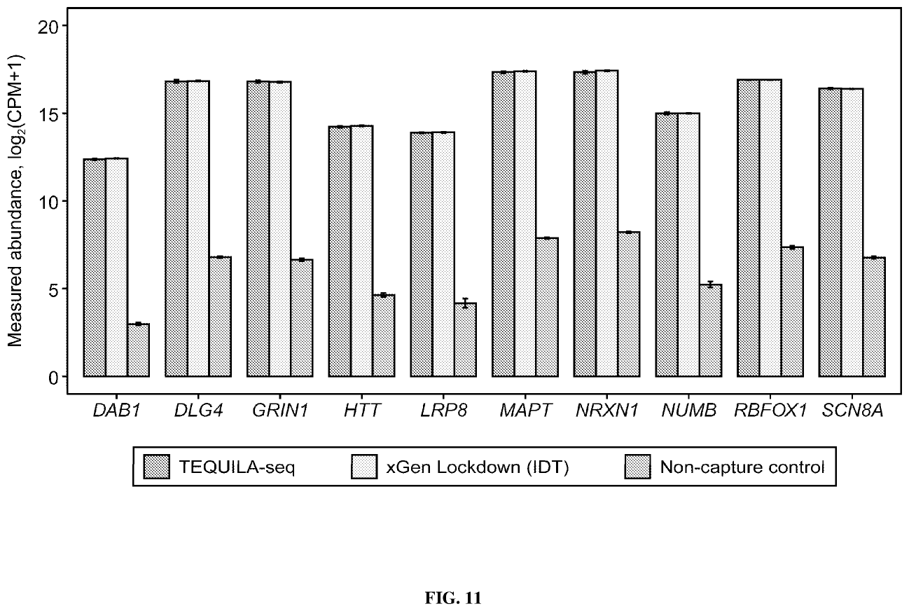

TEQUILA-seq enriches target transcripts comparably to a standard commercial solution. The inventors assessed the capture efficiency and target enrichment of TEQUILA-seq relative to xGen Lockdown probe-based capture sequencing (hereafter referred to as xGen Lockdown-seq), a standard commercial solution for targeted RNA-seq. They initially designed a small test panel of 10 brain genes (DAB1, DLG4, GRIN1, HTT, LRP8, MAPT, NRXN1, NUMB, RBFOX1, and SCN8A). These genes were selected because they are known to express long transcripts with complex AS patterns (Vuong et al., 2016; Wade-Martins, 2012; Sathasivam et al., 2013). For this panel, the inventors synthesized TEQUILA probes and ordered xGen Lockdown probes with the same probe sequences at 1× tiling density. They applied both probe sets to the same human brain cDNA sample and generated nanopore 1D sequencing data (n=3 experimental replicates per probe set) with comparable sequencing depths. Estimated abundances of transcript isoforms were nearly identical across all TEQUILA-seq and xGen Lockdown-seq libraries (FIG. 10). Compared to whole-transcriptome nanopore RNA-seq data generated on the same brain cDNA sample (i.e., a non-capture control), both TEQUILA and xGen Lockdown probes showed comparable performances in enriching transcripts from the 10-gene panel. Specifically, both methods achieved an on-target rate of ˜85% with similar fold enrichment (˜280×) (FIG. 6C). Moreover, both methods yielded nearly identical fold enrichment for each target gene (FIG. 6C, FIG. 11). Collectively, these results demonstrate that TEQUILA-seq achieves comparable performance in capture efficiency to a widely used commercial solution.

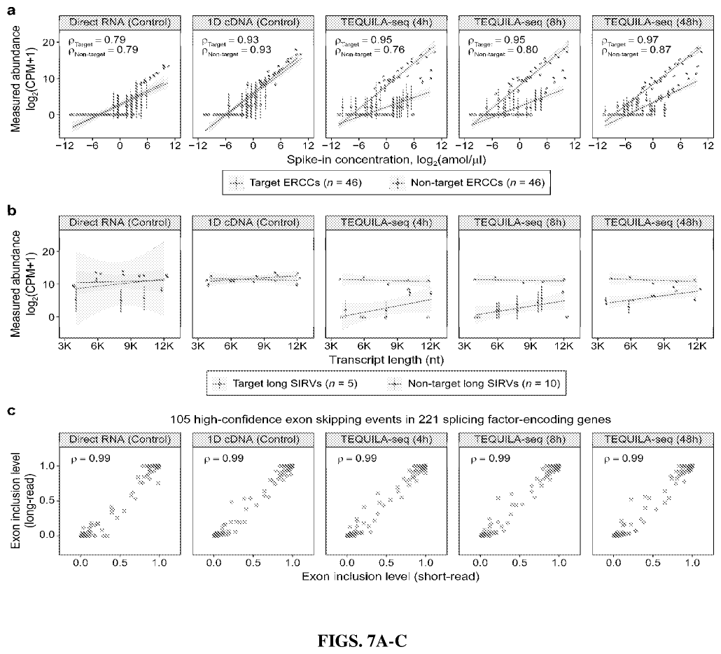

TEQUILA-seq greatly enhances detection and preserves quantification of target transcripts. The inventors assessed the extent to which TEQUILA-seq improves detection of transcript isoforms of target genes by using External RNA Controls Consortium (ERCC) standards. The ERCC standards are 92 synthetic transcripts of unique sequences and their concentrations span six orders of magnitude (Jiang et al., 2011). They synthesized TEQUILA probes for 46 ERCC transcripts covering the entire ERCC concentration range. The remaining 46 ERCCs were not targeted and served as controls. Using TEQUILA-seq, the inventors were able to detect target ERCC transcripts at concentrations as low as 0.18 amol/μl consistently across 3 replicates (≥2 reads per replicate) (FIG. 7A). By contrast, 11.72 amol/ul, a concentration 65.1-fold higher, was the lowest concentration at which they consistently detected target ERCC transcripts by standard nanopore 1D cDNA sequencing (n=3 replicates).

To investigate how the detection sensitivity of TEQUILA-seq changes with sequencing depth, the inventors sequenced TEQUILA-seq libraries prepared from the same ERCC sample for 4 or 8 hours (n=3 replicates per sequencing duration). The 4- and 8-hour TEQUILA-seq runs had sequencing depths that were 6-8 times shallower than the original 48-hour TEQUILA-seq runs. Nevertheless, target ERCC transcripts could still be consistently detected at concentrations as low as 0.18 amol/ul in both the 4- and 8-hour TEQUILA-seq runs. Moreover, estimated abundances of target ERCC transcripts in TEQUILA-seq libraries were highly correlated with their initial spike-in concentrations, even with shallow sequencing depth (Pearson's correlation of 0.97 in 48-hour TEQUILA-seq, and 0.95 in 8-hour and 4-hour TEQUILA-seq). By comparison, the inventors obtained much lower Pearson's correlation values with 1D cDNA sequencing (0.93) and direct RNA sequencing (0.79) (FIG. 7A). These results indicate that the TEQUILA probes enriched all 46 target ERCC transcripts at uniformly elevated levels. By contrast, in the same TEQUILA-seq libraries, the estimated abundances of non-target ERCC transcripts were substantially lower and less correlated (0.76-0.87) with initial spike-in concentrations. Collectively, these results suggest that TEQUILA-seq greatly enhances detection of target transcripts, even for transcripts with low abundances and in samples with shallow sequencing depth.

Next, the inventors examined whether TEQUILA-seq data exhibit any length-dependent biases. They used a set of Spike-In RNA Variants (SIRVs) (Paul et al., 2016) comprising 15 synthetic transcripts of equimolar concentrations that cover transcript lengths from 4,000 to 12,000 nt (hereafter referred to as “long SIRVs”). The inventors synthesized TEQUILA probes for 5 long SIRV transcripts that covered the entire length range of the long SIRV set. They then applied this probe set to RNAs of human SH-SY5Y neuroblastoma cells spiked-in with long SIRVs. All 5 targeted long SIRV transcripts had nearly identical estimated abundances across all TEQUILA-seq run-times when using the library prepared from this sample (FIG. 7B). These results indicate that the TEQUILA probes enrich target transcripts without exhibiting length-dependent biases.

A potential concern with TEQUILA-seq is that different transcript isoforms of a given target gene may not be enriched at equal levels, thus distorting the relative proportions of transcript isoforms. The inventors reasoned that if TEQUILA probes preserve isoform proportions, then transcript inclusion levels of alternatively spliced exons within target genes should remain the same with or without targeted capture. To investigate this issue, they synthesized TEQUILA probes for 221 human genes encoding splicing factors (Han et al., 2013). These 221 genes are known to undergo extensive AS themselves, as a mechanism to regulate splicing factor activity and function (Long & Caceres, 2009; Lareau et al., 2007; Leclair et al., 2020; Dvinge et al., 2016). The inventors applied TEQUILA-seq of this splicing factor gene panel to RNAs of SH-SY5Y cells. For comparison, they also performed bulk short-read RNA-seq, as well as standard nanopore 1D cDNA sequencing and direct RNA sequencing of SH-SY5Y cells.

Across the 221 splicing factor-encoding genes, the estimated transcript inclusion levels of 105 high-confidence exon skipping events (see Methods) were highly correlated between short-read RNA-seq and TEQUILA-seq data (Pearson's correlation of 0.99 at 48-hour, 8-hour, and 4-hour run-times) (FIG. 7C). Similarly, transcript inclusion levels estimated using standard nanopore 1D cDNA or direct RNA sequencing were also highly correlated with estimates made by short-read RNA-seq (Pearson's correlation of 0.99). These results indicate that TEQUILA-seq can preserve the relative proportions of transcript isoforms of target genes.

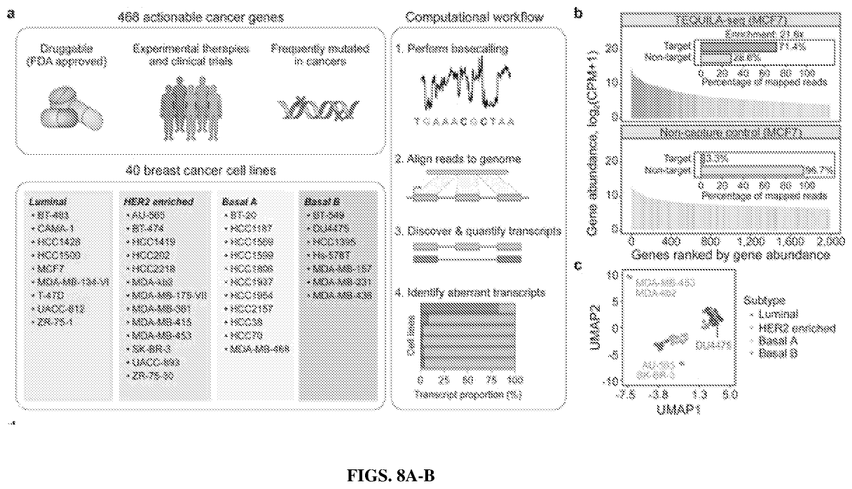

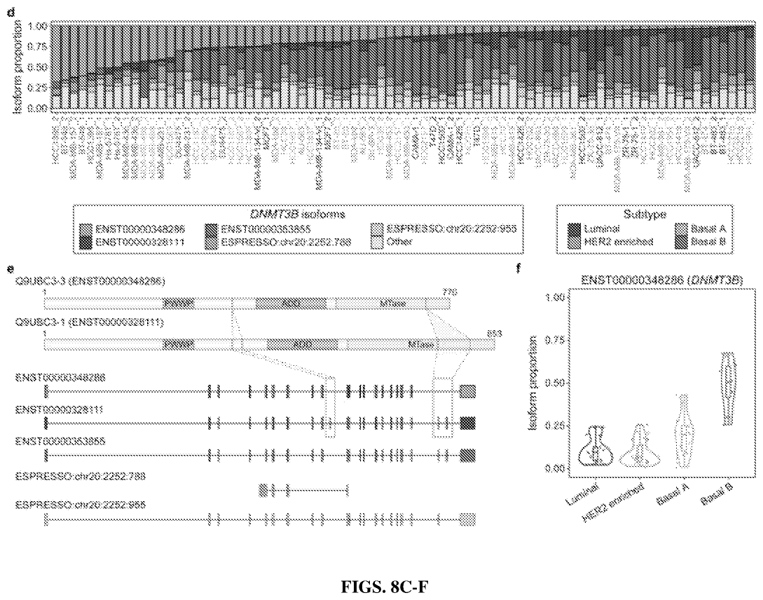

TEQUILA-seq of 468 actionable cancer genes in 40 breast cancer cell lines. To illustrate the biomedical utility of TEQUILA-seq, the inventors performed a TEQUILA-seq analysis of actionable cancer genes in a broad panel of breast cancer cell lines. They synthesized TEQUILA probes for 468 genes interrogated by MSK-IMPACT, an FDA approved diagnostic test for DNA-based mutation profiling of actionable cancer targets (Cheng et al., 2015; Fiala et al., 2021) (FIG. 8A, Supplementary Table 3). As alternative isoform variation is prevalent in breast cancer transcriptomes (Bonnal et al., 2020; Veiga et al., 2022), the inventors hypothesized that a TEQUILA-seq analysis could discover RNA-associated mechanisms and novel aberrant transcript isoforms in breast cancer. They analyzed 40 breast cancer cell lines from the ATCC Breast Cancer Cell Panel representing 4 distinct intrinsic subtypes: luminal, HER2 enriched, basal A, and basal B (FIG. 8A).

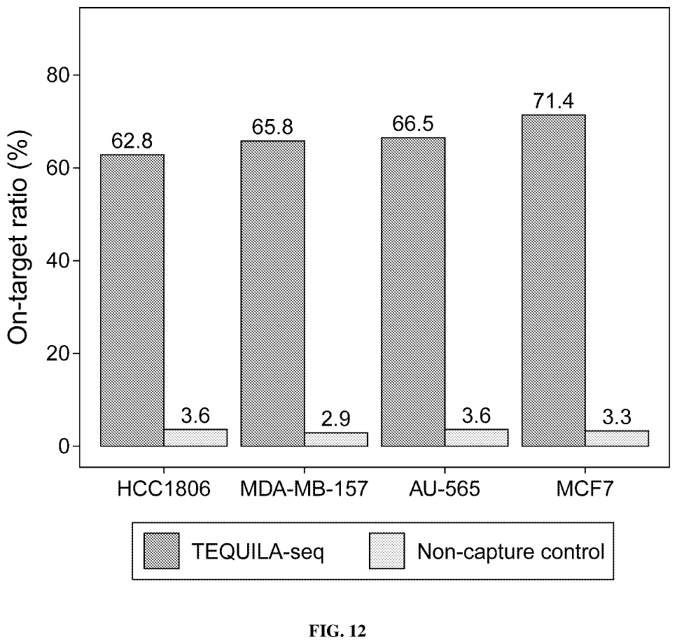

The inventors first assessed the degree to which TEQUILA probes could enrich transcripts of genes in this large 468-gene panel. To this end, they performed TEQUILA-seq and nanopore 1D cDNA sequencing (as a non-capture control) for 4 breast cancer cell lines: MCF7, HCC1806, MDA-MB-157, and AU-565 (FIG. 8B and FIG. 12). On-target rates of the 468 genes in TEQUILA-seq data ranged 62.8% to 71.4%, compared to 2.9% to 3.6% in non-capture control data, demonstrating an average ˜20-fold enrichment. The invetnors then applied TEQUILA-seq to all 40 breast cancer cell lines, with two experimental replicates per cell line, and obtained on-target rates ranging 62.3% to 73.7% across cell lines. Of the 468 genes, 462 were detected (CPM ≥1) in at least one sample (98.7%). From the entire TEQUILA-seq dataset of the 40 cell lines, the inventors discovered 3,122 annotated and 25,519 novel transcript isoforms of the cancer genes. Although many more novel than annotated transcript isoforms were discovered, the majority of reads (79.4% on average across all samples) that mapped to these genes were from annotated transcript isoforms.

Clustering analysis using isoform proportions of the cancer genes revealed two major clusters: cell lines annotated as luminal and HER2-enriched subtypes clustered together, whereas cell lines annotated as basal A and basal B subtypes clustered together (FIG. 8C). Several outlier cell lines were also observed. For instance, pairs of cell lines clustered together as outliers, i.e., MDA-MB-453 and MDA-kb2, as well as AU-565 and SK-BR-3, reflecting the similar cell-line derivation origins (Wilson et al., 2002; Neve et al., 2006). The DU4755 cell line, despite its annotation as the basal B subtype, clustered with the luminal and HER2-enriched subtypes, likely reflecting its controversial subtype classification (Dai et al., 2017; Lehmann et al., 2011).

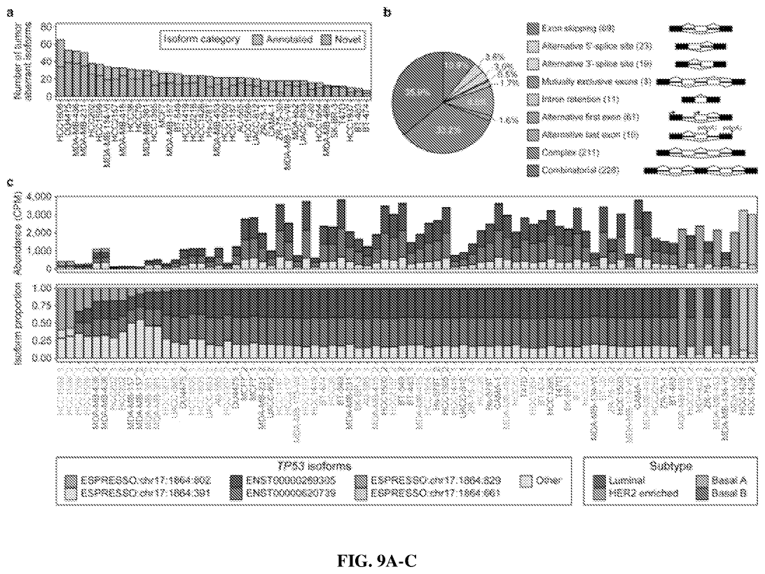

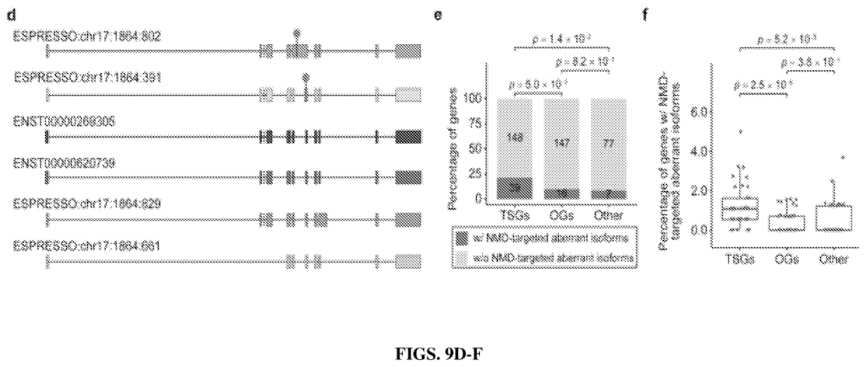

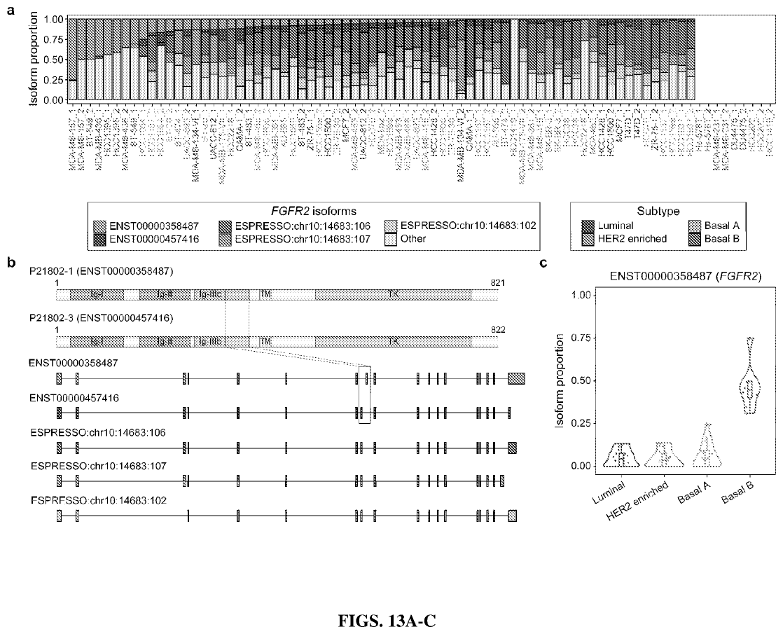

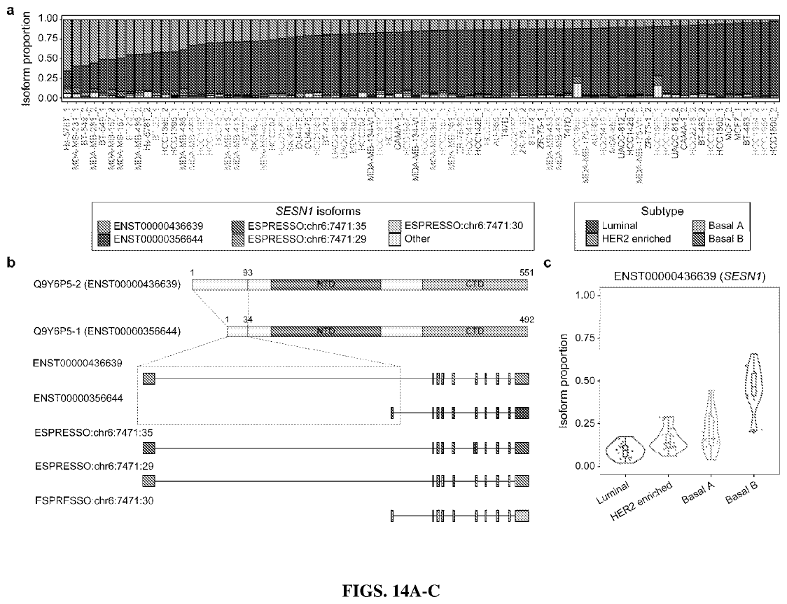

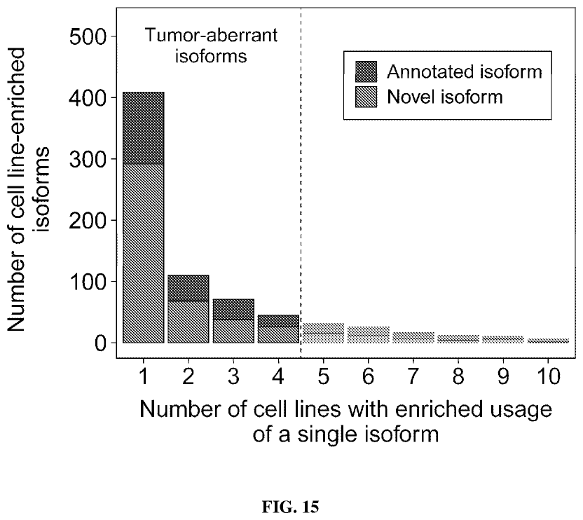

Next, the inventors sought to determine the proportion of transcript isoforms that are associated with different breast cancer intrinsic subtypes (luminal, HER enriched, basal A, basal B) in the 40 breast cancer cell lines (see Methods). For each intrinsic subtype, the inventors compared the mean proportion of a transcript isoform between the subtype-associated cell lines and all other cell lines. At FDR≤0.05, they identified 54 breast cancer subtype-associated transcript isoforms in 50 genes (Supplementary Table 1). As an example, DNMT3B encodes a de novo DNA methyltransferase (Okano et al., 1999; Rhee et al., 2002) These results reveal that an alternative). Compared to the canonical transcript isoform (ENST00000328111), 3 exons (exon 10, 21 and 22) were skipped in the alternative transcript isoform. Skipping of exons 21] and 22 disrupts the C-terminal catalytic domain; the encoded protein isoform is enzymatically inactive (Kastenhuber & Lowe, 2017). To summarize, TEQUILA-seq identified a subtype-associated transcript isoform of DNMT3B, which may have a global effect on DNA methylation of the basal B subtype of breast cancer. Two additional examples of subtype-associated transcript isoforms were shown for FGFR2 (Hafner et al., 2019) (FIGS. 13A-C) and SESN1 (FIGS. 14A-C).Besides identifying subtype-associated transcript isoforms, the inventors also used TEQUILA-seq data to identify “tumor aberrant” transcript isoforms. They define tumor aberrant transcript isoforms as alternative transcript isoforms that are present at significantly elevated proportions in at least one but no more than 4 (i.e., ≤10%) breast cancer cell lines (Methods). In total, the inventors identified 635 aberrant transcript isoforms from 256 genes, with 66.8% being novel transcript isoforms (FIG. 9A, FIG. 15). Comparing aberrant to canonical transcript isoforms of the corresponding genes, the inventors found that transcript isoforms resulting from complex or combinatorial AS events (other than the 7 categories of binary AS events) represented the majority (69.1%) of aberrant transcript isoforms (FIG. 9B). Given that complex or combinatorial AS events are challenging to analyze by short-read RNA-seq (Park et al., 2018), these results highlight the benefit of interrogating the transcript products of actionable cancer genes by long-read RNA-seq.

NMD targeting of aberrant transcript isoforms is a common mechanism of tumor-suppressor gene inactivation. Using TEQUILA-seq data, the inventors identified numerous novel aberrant transcript isoforms in extensively studied cancer genes. The tumor suppressor TP53 encodes a transcription factor involved in regulating diverse cellular processes, such as cell cycle control, DNA repair, apoptosis, metabolism, and cellular senescence (Kastenhuber & Lowe, 2017; Hafner et al., 2019). The inventors discovered a novel aberrant transcript isoform of TP53 (ESPRESSO: chr17:1864:802) as the predominant isoform in the HCC1599 cell line (FIG. 9C). This transcript isoform contains a 568 nt retained intron with respect to the canonical transcript isoform of TP53 (FIG. 9D). The retained intron would introduce an in-frame premature termination codon (PTC), which would target the transcript isoform for degradation via nonsense-mediated mRNA decay (NMD) (Kurosaki et al., 2019). A second, relatively minor novel TP53 transcript isoform (ESPRESSO: chr17:1864:391), which uses a novel 3′ splice site within the retained intron, was also discovered in the HCC1599 cell line (FIG. 9C). This transcript isoform is also NMD-targeted. Overall, the discovery of multiple NMD-targeted transcript isoforms is consistent with the generally low steady-state gene expression level of TP53 in HCC1599, as measured by TEQUILA-seq (FIG. 9C).

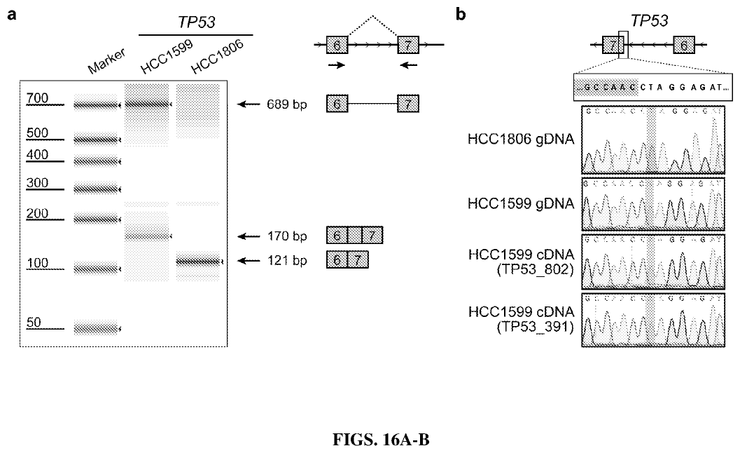

To elucidate the source of these novel TP53 transcript isoforms, the inventors analyzed the whole-genome sequencing (WGS) data of HCC1599 obtained from the Cancer Cell Line Encyclopedia (CCLE). They found that the HCC1599 cell line harbors an A>T somatic mutation next to intron 6 in TP53, and that this mutation disrupts a 3′ splice site at the 3′ end of the retained intron. All WGS reads across this region contain the A>T somatic mutation, as the other allele of TP53 is lost in the tumor genome through loss of heterozygosity (Ghandi et al., 2019). This splice site mutation and resulting transcript products were further confirmed by RT-PCR and Sanger sequencing (FIG. 16A-B). In summary, TEQUILA-seq discovered novel aberrant transcript isoforms of TP53 in HCC1599, which may contribute to inactivating TP53 in this cell line.

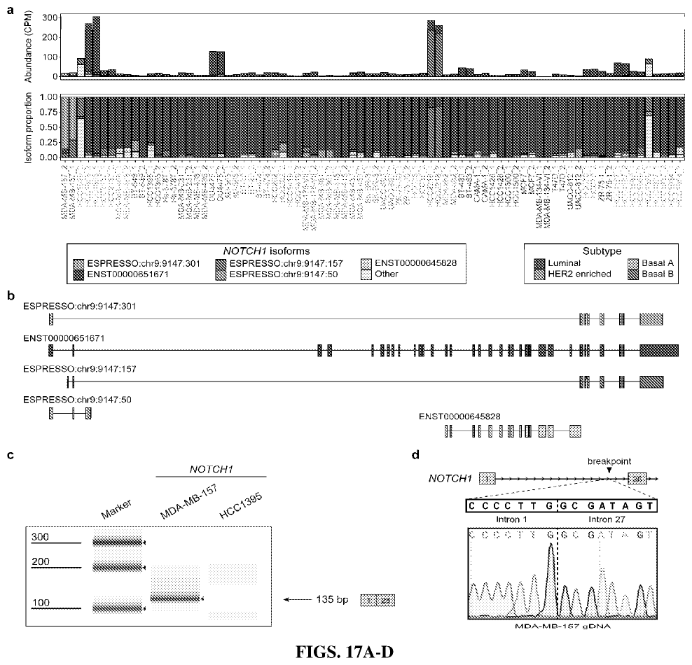

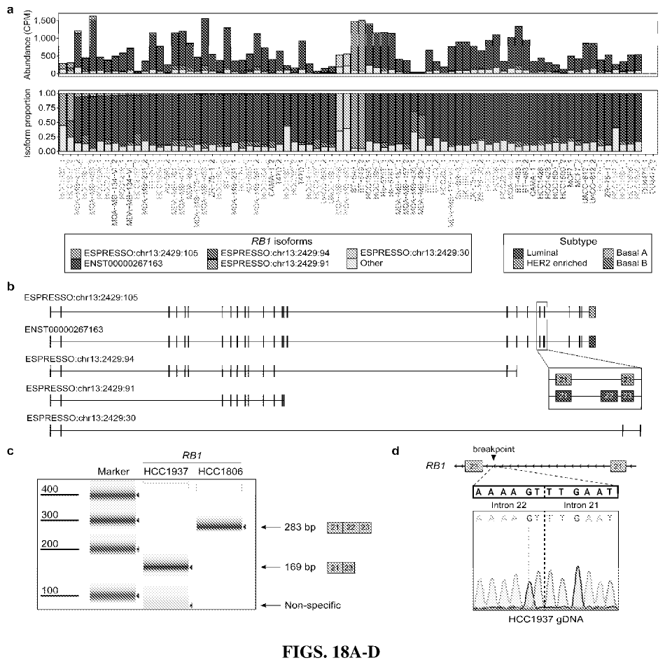

Additionally, the inventors discovered aberrant transcript isoforms of multiple other genes encoding tumor suppressors, such as NOTCH1 and RB1. A novel aberrant transcript isoform of NOTCH1 (ESPRESSO: chr9: 9147:301) was found as the predominant transcript isoform in the MDA-MB-157 cell line. This transcript isoform lacks the segment spanning exons 2 to 27 with respect to the canonical transcript isoform of NOTCH1 (FIGS. 17A-D). In the HCC1937 cell line, the inventors discovered a novel aberrant transcript isoform of RB1 (ESPRESSO: chr13:2429:105), which lacks exon 22 with respect to the canonical transcript isoform (FIGS. 18A-D). Using RT-PCR and Sanger sequencing, they confirmed that the novel aberrant transcript isoforms result from focal genomic deletions that deleted multiple exons (in NOTCH1) or one exon (in RB1) from the tumor genome (FIGS. 17A-D and 18A-D).

The discovery of NMD-targeted aberrant transcript isoforms in TP53 raises an interesting question of whether this observation represents a recurring RNA-associated mechanism for inactivating tumor suppressor genes in breast cancer. To address this question, the inventors categorized the 468 cancer genes analyzed by TEQUILA-seq into three groups: 196 tumor-suppressor genes (TSGs), 179 oncogenes (OGs), and 93 “Other” genes. Among genes expressed in at least 10 of the 40 breast cancer cell lines (i.e., average CPM of 2 replicates ≥1), NMD-targeted aberrant transcript isoforms were significantly more enriched in TSGs (20.9% in TSGs, 9.8% in OGs, and 8.3% in Other; FIG. 9E). Additionally, the percentages of genes with NMD-targeted aberrant transcript isoforms among genes detected in each of the 40 breast cancer cell lines were significantly higher for TSGs than for OGs and Other genes (two-sided paired Wilcoxon test; FIG. 9E). These results suggest that aberrant alternative isoform variation coupled with NMD represents a common mechanism for inactivating TSGs in individual tumors.

### Example 5—Discussion

Targeted capture followed by long-read RNA-seq offers a powerful strategy to perform focused analyses of transcript isoforms for preselected gene panels. It leverages the ability of long-read sequencing platforms to sequence full-length transcript molecules end-to-end, while circumventing their weaknesses of limited sequencing yield and low transcript coverage. Nevertheless, existing solutions for targeted long-read RNA-seq are either expensive (Lagarde et al., 2017), or difficult to set up and implement (Sheynkman et al., 2020). Here, the inventors present TEQUILA-seq, a new method for targeted long-read RNA-seq. The TEQUILA process for synthesizing biotinylated capture oligos is versatile, easy to implement, and highly cost-effective. Non-biotinylated oligo templates as starting material can be acquired as an array-synthesized oligo pool at modest cost from various commercial vendors. By using nickase-triggered isothermal SDA, the TEQUILA process can generate large quantities of biotinylated capture oligos from limited starting material, enabling a large number (>10,000) of capture reactions. As the nickase releases the synthesized strand from the universal adaptor sequence, the TEQUILA probes are free of any artificial adaptor sequence, with only complementary sequences against the targeted sequences. TEQUILA reduces the initial set up cost and dramatically reduces the per-reaction cost of targeted capture by 2-3 orders of magnitude, as compared to a standard commercial solution (Supplementary Tables 1 and 2). With this cost structure, TEQUILA-seq can practically scale up to large cohorts with many biological samples.

The inventors performed TEQUILA-seq of both synthetic RNAs and human mRNAs, using multiple gene panels ranging in size from a small panel of 10 brain genes to a large panel of 468 actionable cancer genes. The inventors' comprehensive benchmark analyses indicate consistently high on-target rate and fold enrichment across all samples and gene panels analyzed. Using synthetic RNAs with known transcript structures and concentrations, the inventors showed that TEQUILA-seq can substantially improve the sensitivity of detecting low-abundance transcripts. At the same time, the estimated abundances of target transcripts based on TEQUILA-seq data correlated highly with the ground truth (FIG. 7A). They also showed that TEQUILA-seq data do not exhibit length-dependent biases in transcript detection and quantification (FIG. 7B). Moreover, by comparing TEQUILA-seq data of a human gene panel to deep short-read RNA-seq data on the same sample, the inventors showed that TEQUILA-seq can preserve transcript isoform proportions of target genes (FIG. 7C). Overall, these results indicate that TEQUILA-seq provides a robust tool for transcript discovery and quantification for target genes.

Targeted sequencing or WGS of tumor DNA has been broadly used in research and clinical settings (Cheng et al., 2015; Fiala et al., 2021; Chakravarty & Solit, 2021; Staaf et al., 2019). However, RNA-level dysregulation is prevalent in cancer transcriptomes (Pan et al., 2021), and recent studies have established the complementary value of transcriptome sequencing for cancer genomic profiling (Beaubier et al., 2019; Horak, et al., 2021; Shukla et al., 2022). By performing TEQUILA-seq of 468 actionable cancer genes across a broad panel of 40 breast cancer cell lines, the inventors discovered numerous known or novel transcript isoforms with potential functional relevance. For example, they found that an alternative transcript isoform of DNMT3B, lacking 2 exons that encode part of its C-terminal catalytic domain, is highly enriched in basal B breast cancer cell lines (FIGS. 8D, 8F). This finding has implications for the epigenetic regulation and DNA methylome of the basal B subtype, the most aggressive subtype of breast cancer (Harbeck et al., 2019; Bianchini et al., 2022). The inventors also discovered novel aberrant transcript isoforms of multiple genes encoding tumor suppressors, such as TP53, NOTCH1, and RB1 (FIGS. 9D, 9D; FIGS. 17A-D and 18A-D). Using the full-length transcript information provided by TEQUILA-seq, they can infer the function of isoform variation as it relates to transcript and protein products. For example, the aberrant transcript isoforms of TP53 discovered in HCC1599 cell line would introduce an in-frame PTC and trigger transcript degradation via the NMD pathway. Expanding this analysis to all aberrant transcript isoforms discovered in the breast cancer dataset, the inventors found that TSGs are significantly more enriched for NMD-targeted aberrant transcript isoforms, as compared to OGs and other cancer genes (FIGS. 9E-F). Thus, the TEQUILA-seq analysis reveals a common mechanism for inactivating TSGs in cancer cells, via aberrant alternative isoform variation coupled with transcript degradation via NMD.

The inventors envision that TEQUILA-seq may facilitate broad applications of targeted long-read RNA-seq in diverse biomedical settings. Here, the inventors illustrated a proof-of-concept application of TEQUILA-seq to cancer genes; however, TEQUILA-seq can be applied to any gene panel of interest for focused discovery and quantification of transcript isoforms. For example, TEQUILA-seq of genes implicated in a given category of Mendelian genetic diseases can be used for RNA-guided genetic diagnosis (Cummings et al., 2017). Likewise, TEQUILA-seq of genes involved in oncogenic gene fusions can be used for discovering actionable fusion transcripts for precision oncology applications (Reeser et al., 2017; Heyer et al., 2019). Beyond targeted RNA-seq, TEQUILA probes can also be used for various applications related to targeted DNA sequencing, such as targeted analysis of DNA methylation (Deng et al., 2009; Liu et al., 2020) and chromatin conformation (Hughes et al., 2014; McCord et al., 2020).

SUPPLEMENTARY TABLE 1
Reagent Costs for Synthesizing TEQUILA Probes

| | | | | | | | Cost per | |
|---|---|---|---|---|---|---|---|---|
| | | | | | | | probe | *Cost per |
| | | | | | | | synthesis | capture |
| | | Catalog | | | Pricing | Number of | reaction | reaction |
| Reagent | Manufacturer | number | Size | | ($) | reactions | ($) | ($) |
| Biotin-16-Aminoallyl-2′-dUTP | Trilink | N-5001-1 | 1 | μmol | 655.00 | 100 | 6.55 | 0.07 |
| Deoxynucleotide (dNTP) Solution Set | NEB | N0446 | 4 × 0.25 ml, 100 mM | | 132.44 | 600 | 0.22 | 0.00 |
| Strand Displacement Amplification (SDA) Primer | IDT | — | 25 | nmol | 11.00 | 24,000 | 0.00 | 0.00 |
| Dithiothreitol (DTT) | Thermo Fisher | 707265ML | 5 ml, 0.1M | | 105.00 | 5,000 | 0.02 | 0.00 |
| T4 Gene 32 Protein | NEB | M0300S | 100 | μg | 72.00 | 10 | 7.20 | 0.07 |
| Klenow Fragment (3′→5′ exo-) | NEB | M0212M | 1,000 | units | 226.80 | 25 | 9.07 | 0.09 |
| Nt.BspQl | NEB | R0644S | 1,000 | units | 63.90 | 50 | 1.28 | 0.01 |

| | | |
|---|---|---|
| NEBuffer 3.1 | NEB | 1 × 1.25 ml, 10X |

| | | |
|---|---|---|
| Total cost per reaction | 24.34 | 0.24 |

*Cost per capture reaction was calculated with the assumption that probes generated from one TEQUILA probe synthesis reaction are sufficient for 100 capture reactions (one probe synthesis reaction starting with 2 ng of oligo pool templates can generate at least 10 μg of probes, and one capture reaction requires 100 ng of TEQUILA probes).

SUPPLEMENTARY TABLE 2
Cost Comparison Between IDT xGen Lockdown
Probes and TEQUILA Probes
IDT xGen Lockdown probe pool

| | | |
|---|---|---|
| Panel size | Pricing | Cost per capture reaction |

16 reactions

| | | |
|---|---|---|
| 50 to 1,000 probes | $5.00 per probe | $15.63 to $312.50 |
| 1,001 to 2,000 probe [symbol] | $5,000.00 | $312.50 |
| 2.001 to 3,000 probe [symbol] | $6,500.00 | $406.25 |
| 3,001 to 4,000 probe [symbol] | $9,000.00 | $562.50 |
| 4,001 to 5,000 probe [symbol] | $11,000.00 | $687.50 |
| 5,001 to 6,000 probe [symbol] | $13,000.00 | $812.50 |
| >6,000 probes | Inquire for price | NA |

96 reactions

| | | |
|---|---|---|
| 50 to 2,000 probes | $9.00 per probe | $4.69 to $187.50 |
| 2,001 to 3,000 probe [symbol] | $18,000.00 | $187.50 |
| 3,001 to 4,000 probe [symbol] | $24,000.00 | $250.00 |
| 4.001 to 6,000 probe [symbol] | $30,000.00 | $312.50 |
| 6,001 to 8,000 probe [symbol] | $36,000.00 | $375.00 |
| >8,000 probes | Inquire for price | NA |

4 × 96 reactions

| | | |
|---|---|---|
| 50 to 4,000 probes | $12.00 per probe | $1.56 to $125.00 |
| 4,001 to 5,000 probe [symbol] | $48,000.00 | $125.00 |
| 5.001 to 7,000 probe [symbol] | $60,000.00 | $156.25 |
| 7,001 to 8,000 probe [symbol] | $72,000.00 | $187.50 |
| >8,000 probes | Inquire for price | NA |

Twist Bioscience oligo pool for TEQUILA probe synthesis
~10,000 reactions

| | | Cost per capture reaction | |
|---|---|---|---|
| | | Oligo cost per | Reagent cost |
| Panel size | Pricing | reaction | included |
| 101 to 500 oligos | $606.45 | $0.06 | $0.30 |
| 501 to 1,000 oligos | $910.00 | $0.09 | $0.33 |
| 1,001 to 2,000 oligos | $1,213.55 | $0.12 | $0.36 |
| 2,001 to 6,000 oligos | $1,820.00 | $0.18 | $0.43 |
| 6,001 to 12,000 oligos | $2,433.60 | $0.24 | $0.49 |
| 12,001 to 18,000 oligo [symbol] | $3,163.55 | $0.32 | $0.56 |
| 18,001 to 24,000 oligo [symbol] | $4,112.55 | $0.41 | $0.65 |
| 24,001 to 30,000 oligo [symbol] | $5,346.25 | $0.53 | $0.78 |

Note:
The maximum number of capture reactions using TEQUILA probes was calculated with the assumption that the oligo pool from Twist Bioscience is sufficient for at least 100 probe synthesis reactions, and probes generated from one TEQUILA probe synthesis reaction are sufficient for 100 capture reactions. [symbol]
indicates data missing or illegible when filed

SUPPLEMENTAL TABLE 3
Panel of 468 Actionable Cancer-Associated Genes

| HGNC Gene Symbol | Ensembl Gene ID |
|---|---|
| ABL1 | ENSG00000097007 |
| ABRAXAS1 | ENSG00000163322 |
| ACVR1 | ENSG00000115170 |
| AGO2 | ENSG00000123908 |
| AKT1 | ENSG00000142208 |
| AKT2 | ENSG00000105221 |
| AKT3 | ENSG00000117020 |
| ALK | ENSG00000171094 |
| ALOX12B | ENSG00000179477 |
| AMER1 | ENSG00000184675 |
| ANKRD11 | ENSG00000167522 |
| APC | ENSG00000134982 |
| AR | ENSG00000169083 |
| ARAF | ENSG00000078061 |
| ARID1A | ENSG00000117713 |
| ARID1B | ENSG00000049618 |
| ARID2 | ENSG00000189079 |
| ARID5B | ENSG00000150347 |
| ASXL1 | ENSG00000171456 |
| ASXL2 | ENSG00000143970 |
| ATM | ENSG00000149311 |
| ATR | ENSG00000175054 |
| ATRX | ENSG00000085224 |
| AURKA | ENSG00000087586 |
| AURKB | ENSG00000178999 |
| AXIN1 | ENSG00000103126 |
| AXIN2 | ENSG00000168646 |
| AXL | ENSG00000167601 |
| B2M | ENSG00000166710 |
| BABAM1 | ENSG00000105393 |
| BAP1 | ENSG00000163930 |
| BARD1 | ENSG00000138376 |
| BBC3 | ENSG00000105327 |
| BCL10 | ENSG00000142867 |
| BCL2 | ENSG00000171791 |
| BCL2L1 | ENSG00000171552 |
| BCL2L11 | ENSG00000153094 |
| BCL6 | ENSG00000113916 |
| BCOR | ENSG00000183337 |
| BIRC3 | ENSG00000023445 |
| BLM | ENSG00000197299 |
| BMPR1A | ENSG00000107779 |
| BRAF | ENSG00000157764 |
| BRCA1 | ENSG00000012048 |
| BRCA2 | ENSG00000139618 |
| BRD4 | ENSG00000141867 |
| BRIP1 | ENSG00000136492 |
| BTK | ENSG00000010671 |
| CALR | ENSG00000179218 |
| CARD11 | ENSG00000198286 |
| CARM1 | ENSG00000142453 |
| CASP8 | ENSG00000064012 |
| CBFB | ENSG00000067955 |
| CBL | ENSG00000110395 |
| CCND1 | ENSG00000110092 |
| CCND2 | ENSG00000118971 |
| CCND3 | ENSG00000112576 |
| CCNE1 | ENSG00000105173 |
| CCNQ | ENSG00000262919 |
| CD274 | ENSG00000120217 |
| CD276 | ENSG00000103855 |
| CD79A | ENSG00000105369 |
| CD79B | ENSG00000007312 |
| CDC42 | ENSG00000070831 |
| CDC73 | ENSG00000134371 |
| CDH1 | ENSG00000039068 |
| CDK12 | ENSG00000167258 |
| CDK4 | ENSG00000135446 |
| CDK6 | ENSG00000105810 |
| CDK8 | ENSG00000132964 |
| CDKN1A | ENSG00000124762 |
| CDKN1B | ENSG00000111276 |
| CDKA2A | ENSG00000147889 |
| CDKN2B | ENSG00000147883 |
| CDKN2C | ENSG00000123080 |
| CEBPA | ENSG00000245848 |
| CENPA | ENSG00000115163 |
| CHEK1 | ENSG00000149554 |
| CHEK2 | ENSG00000183765 |
| CIC | ENSG00000079432 |
| COP1 | ENSG00000143207 |
| CREBBP | ENSG00000005339 |
| CRKL | ENSG00000099942 |
| CRLF2 | ENSG00000205755 |
| CSDE1 | ENSG00000009307 |
| CSF1R | ENSG00000182578 |
| CSF3R | ENSG00000119535 |
| CTCF | ENSG00000102974 |
| CTLA4 | ENSG00000163599 |
| CTNNB1 | ENSG00000168036 |
| CUL3 | ENSG00000036257 |
| CXCR4 | ENSG00000121966 |
| CYLD | ENSG00000083799 |
| CYSLTR2 | ENSG00000152207 |
| DAXX | ENSG00000204209 |
| DCUN1D1 | ENSG00000043093 |
| DDR2 | ENSG00000162733 |
| DICER1 | ENSG00000100697 |
| DIS3 | ENSG00000083520 |
| DNAJB1 | ENSG00000132002 |
| DNMT1 | ENSG00000130816 |
| DNMT3A | ENSG00000119772 |
| DNMT3B | ENSG00000088305 |
| DOT1L | ENSG00000104885 |
| DROSHA | ENSG00000113360 |
| DUSP4 | ENSG00000120875 |
| E2F3 | ENSG00000112242 |
| EED | ENSG00000074266 |
| EGFL7 | ENSG00000172889 |
| EGFR | ENSG00000146648 |
| EIF1AX | ENSG00000173674 |
| EIF4A2 | ENSG00000156976 |
| EIF4E | ENSG00000151247 |
| ELF3 | ENSG00000163435 |
| ELOC | ENSG00000154582 |
| EP300 | ENSG00000100393 |
| EPAS1 | ENSG00000116016 |
| EPCAM | ENSG00000119888 |
| EPHA3 | ENSG00000044524 |
| EPHA5 | ENSG00000145242 |
| EPHA7 | ENSG00000135333 |
| EPHB1 | ENSG00000154928 |
| ERBB2 | ENSG00000141736 |
| ERBB3 | ENSG00000065361 |
| ERBB4 | ENSG00000178568 |
| ERCC2 | ENSG00000104884 |
| ERCC3 | ENSG00000163161 |
| ERCC4 | ENSG00000175595 |
| ERCC5 | ENSG00000134899 |
| ERF | ENSG00000105722 |
| ERG | ENSG00000157554 |
| ERRFI1 | ENSG00000116285 |
| ESR1 | ENSG00000091831 |
| ETV1 | ENSG00000006468 |
| ETV6 | ENSG00000139083 |
| EZH1 | ENSG00000108799 |
| EZH2 | ENSG00000106462 |
| FANCA | ENSG00000187741 |
| FANCC | ENSG00000158169 |
| FAT1 | ENSG00000083857 |
| FBXW7 | ENSG00000109670 |
| FGF19 | ENSG00000162344 |
| FGF3 | ENSG00000186895 |
| FGF4 | ENSG00000075388 |
| FGFR1 | ENSG00000077782 |
| FGFR2 | ENSG00000066468 |
| FGFR3 | ENSG00000068078 |
| FGFR4 | ENSG00000160867 |
| FH | ENSG00000091483 |
| FLCN | ENSG00000154803 |
| FLT1 | ENSG00000102755 |
| FLT3 | ENSG00000122025 |
| FLT4 | ENSG00000037280 |
| FOXA1 | ENSG00000129514 |
| FOXL2 | ENSG00000183770 |
| FOXO1 | ENSG00000150907 |
| FOXP1 | ENSG00000114861 |
| FUBP1 | ENSG00000162613 |
| FYN | ENSG00000010810 |
| GATA1 | ENSG00000102145 |
| GATA2 | ENSG00000179348 |
| GATA3 | ENSG00000107485 |
| GLI1 | ENSG00000111087 |
| GNA11 | ENSG00000088256 |
| GNAQ | ENSG00000156052 |
| GNAS | ENSG00000087460 |
| GPS2 | ENSG00000132522 |
| GREM1 | ENSG00000166923 |
| GRIN2A | ENSG00000183454 |
| GSK3B | ENSG00000082701 |
| H1-2 | ENSG00000187837 |
| H2BC5 | ENSG00000158373 |
| H3-3A | ENSG00000163041 |
| H3-3B | ENSG00000132475 |
| H3-4 | ENSG00000168148 |
| H3-5 | ENSG00000188375 |
| H3C1 | ENSG00000275714 |
| H3C10 | ENSG00000278828 |
| H3C11 | ENSG00000275379 |
| H3C12 | ENSG00000197153 |
| H3C13 | ENSG00000183598 |
| H3C14 | ENSG00000203811 |
| H3C2 | ENSG00000286522 |
| H3C3 | ENSG00000287080 |
| H3C4 | ENSG00000197409 |
| H3C6 | ENSG00000274750 |
| H3C7 | ENSG00000277775 |
| H3C8 | ENSG00000273983 |
| HGF | ENSG00000019991 |
| HLA-A | ENSG00000206503 |
| HLA-B | ENSG00000234745 |
| HNF1A | ENSG00000135100 |
| HOXB13 | ENSG00000159184 |
| HRAS | ENSG00000174775 |
| ICOSLG | ENSG00000160223 |
| ID3 | ENSG00000117318 |
| IDH1 | ENSG00000138413 |
| IDH2 | ENSG00000182054 |
| IFNGR1 | ENSG00000027697 |
| IGF1 | ENSG00000017427 |
| IGF1R | ENSG00000140443 |
| IGF2 | ENSG00000167244 |
| IKKE | ENSG00000263528 |
| IKZF1 | ENSG00000185811 |
| IL10 | ENSG00000136634 |
| IL7R | ENSG00000168685 |
| INHA | ENSG00000123999 |
| INHBA | ENSG00000122641 |
| INPP4A | ENSG00000040933 |
| INPP4B | ENSG00000109452 |
| INPPL1 | ENSG00000165458 |
| INSR | ENSG00000171105 |
| IRF4 | ENSG00000137265 |
| IRS1 | ENSG00000169047 |
| IRS2 | ENSG00000185950 |
| JAK1 | ENSG00000162434 |
| JAK2 | ENSG00000096968 |
| JAK3 | ENSG00000105639 |
| JUN | ENSG00000177606 |
| KDM5A | ENSG00000073614 |
| KDM5C | ENSG00000126012 |
| KDM6A | ENSG00000147050 |
| KDR | ENSG00000128052 |
| KEAP1 | ENSG00000079999 |
| KIT | ENSG00000157404 |
| KLF4 | ENSG00000136826 |
| KMT2A | ENSG00000118058 |
| KMT2B | ENSG00000272333 |
| KMT2C | ENSG00000055609 |
| KMT2D | ENSG00000167548 |
| KMT5A | ENSG00000183955 |
| KNSTRN | ENSG00000128944 |
| KRAS | ENSG00000133703 |
| LATS1 | ENSG00000131023 |
| LATS2 | ENSG00000150457 |
| LMO1 | ENSG00000166407 |
| LYN | ENSG00000254087 |
| MALT1 | ENSG00000172175 |
| MAP2K1 | ENSG00000169032 |
| MAP2K2 | ENSG00000126934 |
| MAP2K4 | ENSG00000065559 |
| MAP3K1 | ENSG00000095015 |
| MAP3K13 | ENSG00000073803 |
| MAP3K14 | ENSG00000006062 |
| MAPK1 | ENSG00000100030 |
| MARK3 | ENSG00000102882 |
| MAPKAP1 | ENSG00000119487 |
| MAX | ENSG00000125952 |
| MCL1 | ENSG00000143384 |
| MDC1 | ENSG00000137337 |
| MDM2 | ENSG00000135679 |
| MDM4 | ENSG00000198625 |
| MED12 | ENSG00000184634 |
| MEF2B | ENSG00000213999 |
| MEN1 | ENSG00000133895 |
| MET | ENSG00000105976 |
| MGA | ENSG00000174197 |
| MITF | ENSG00000187098 |
| MLH1 | ENSG00000076242 |
| MPL | ENSG00000117400 |
| MRE11 | ENSG00000020922 |
| MSH2 | ENSG00000095002 |
| MSH3 | ENSG00000113318 |
| MSH6 | ENSG00000116062 |
| MSI1 | ENSG00000135097 |
| MSI2 | ENSG00000153944 |
| MST1 | ENSG00000173531 |
| MST1R | ENSG00000164078 |
| MTOR | ENSG00000198793 |
| MUTYH | ENSG00000132781 |
| MYC | ENSG00000136997 |
| MYCL | ENSG00000116990 |
| MYCN | ENSG00000134323 |
| MYD88 | ENSG00000172936 |
| MYOD1 | ENSG00000129152 |
| NBN | ENSG00000104320 |
| NCOA3 | ENSG00000124151 |
| NCOR1 | ENSG00000141027 |
| NEGR1 | ENSG00000172260 |
| NF1 | ENSG00000196712 |
| NF2 | ENSG00000186575 |
| NFE2L2 | ENSG00000116044 |
| NFKBIA | ENSG00000100906 |
| NKX2-1 | ENSG00000136352 |
| NKX3-1 | ENSG00000167034 |
| NOTCH1 | ENSG00000148400 |
| NOTCH2 | ENSG00000134250 |
| NOTCH3 | ENSG00000074181 |
| NOTCH4 | ENSG00000204301 |
| NPM1 | ENSG00000181163 |
| NRAS | ENSG00000213281 |
| NSD1 | ENSG00000165671 |
| NSD2 | ENSG00000109685 |
| NSD3 | ENSG00000147548 |
| NTHL1 | ENSG00000065057 |
| NTRK1 | ENSG00000198400 |
| NTRK2 | ENSG00000148053 |
| NTRK3 | ENSG00000140538 |
| NUF2 | ENSG00000143228 |
| NUP93 | ENSG00000102900 |
| PAK1 | ENSG00000149269 |
| PAK5 | ENSG00000101349 |
| PAL82 | ENSG00000083093 |
| PARP1 | ENSG00000143799 |
| PAX5 | ENSG00000196092 |
| PBRM1 | ENSG00000163939 |
| PDCD1 | ENSG00000188389 |
| PDCD1LG2 | ENSG00000197646 |
| PDGFRA | ENSG00000134853 |
| PDGFRB | ENSG00000113721 |
| PDPK1 | ENSG00000140992 |
| PGR | ENSG00000082175 |
| PHOX2B | ENSG00000109132 |
| PIK3C2G | ENSG00000139144 |
| PIK3C3 | ENSG00000078142 |
| PIK3CA | ENSG00000121879 |
| PIK3CB | ENSG00000051382 |
| PIK3CD | ENSG00000171608 |
| PIK3CG | ENSG00000105851 |
| PIK3R1 | ENSG00000145675 |
| PIK3R2 | ENSG00000105647 |
| PIK3R3 | ENSG00000117461 |
| PIM1 | ENSG00000137193 |
| PLCG2 | ENSG00000197943 |
| PLK2 | ENSG00000145632 |
| PMAIP1 | ENSG00000141682 |
| PMS1 | ENSG00000064933 |
| PMS2 | ENSG00000122512 |
| PNRC1 | ENSG00000146278 |
| POLD1 | ENSG00000062822 |
| POLE | ENSG00000177084 |
| PPARG | ENSG00000132170 |
| PPM1D | ENSG00000170836 |
| PPP2R1A | ENSG00000105568 |
| PPP4R2 | ENSG00000163605 |
| PPP6C | ENSG00000119414 |
| PROM1 | ENSG00000057657 |
| PROM14 | ENSG00000147596 |
| PREX2 | ENSG00000046889 |
| PRKAR1A | ENSG00000108946 |
| PRKCI | ENSG00000163558 |
| PRKD1 | ENSG00000184304 |
| PRKN | ENSG00000185345 |
| PTCH1 | ENSG00000185920 |
| PTEN | ENSG00000171862 |
| PTP4A1 | ENSG00000112245 |
| PTPN11 | ENSG00000179295 |
| PTPRD | ENSG00000153707 |
| PTPRS | ENSG00000105426 |
| PTPRT | ENSG00000196090 |
| RAB35 | ENSG00000111737 |
| RAC1 | ENSG00000136238 |
| RAC2 | ENSG00000128340 |
| RAD21 | ENSG00000164754 |
| RAD50 | ENSG00000113522 |
| RAD51 | ENSG00000051180 |
| RAD51B | ENSG00000182185 |
| RAD51C | ENSG00000108384 |
| RAD51D | ENSG00000185379 |
| RAD52 | ENSG00000002016 |
| RAD54L | ENSG00000085999 |
| RAF1 | ENSG00000132155 |
| RARA | ENSG00000131759 |
| RASA1 | ENSG00000145715 |
| RB1 | ENSG00000139687 |
| RBM10 | ENSG00000182872 |
| RECQL | ENSG00000004700 |
| RECQL4 | ENSG00000160957 |
| REL | ENSG00000162924 |
| RET | ENSG00000165731 |
| RHEB | ENSG00000106615 |
| RHOA | ENSG00000067560 |
| RICTOR | ENSG00000164327 |
| RIT1 | ENSG00000143622 |
| RNF43 | ENSG00000108375 |
| ROS1 | ENSG00000047936 |
| RPS6KA4 | ENSG00000162302 |
| RPS6KB2 | ENSG00000175634 |
| RPTOR | ENSG00000141564 |
| RRAGC | ENSG00000116954 |
| RRAS | ENSG00000126458 |
| RRAS2 | ENSG00000133818 |
| RTEL1 | ENSG00000258366 |
| RUNX1 | ENSG00000159216 |
| RXRA | ENSG00000186350 |
| RYBP | ENSG00000163602 |
| SDHA | ENSG00000073578 |
| SDHAF2 | ENSG00000167985 |
| SDHB | ENSG00000117118 |
| SDHC | ENSG00000143252 |
| SDHD | ENSG00000204370 |
| SESN1 | ENSG00000080546 |
| SESN2 | ENSG00000130766 |
| SESN3 | ENSG00000149212 |
| SETD2 | ENSG00000181555 |
| SF3B1 | ENSG00000115524 |
| SH2B3 | ENSG00000111252 |
| SH2D14 | ENSG00000183918 |
| SHOC2 | ENSG00000108061 |
| SHQ1 | ENSG00000144736 |
| SLX4 | ENSG00000188827 |
| SMAD2 | ENSG00000175387 |
| SMAD3 | ENSG00000166949 |
| SMAD4 | ENSG00000141646 |
| SMARCA4 | ENSG00000127616 |
| SMARCB1 | ENSG00000099956 |
| SMARCD1 | ENSG00000066117 |
| SMO | ENSG00000128602 |
| SMYD3 | ENSG00000185420 |
| SOCS1 | ENSG00000185338 |
| SOS1 | ENSG00000115904 |
| SOX17 | ENSG00000164736 |
| SOX2 | ENSG00000181449 |
| SOX9 | ENSG00000125398 |
| SPEN | ENSG00000065526 |
| SPOP | ENSG00000121067 |
| SPRED1 | ENSG00000166068 |
| SRC | ENSG00000197122 |
| SRSF2 | ENSG00000161547 |
| STAG2 | ENSG00000101972 |
| STAT3 | ENSG00000168610 |
| STAT5A | ENSG00000126561 |
| STAT5B | ENSG00000173757 |
| STK11 | ENSG00000118046 |
| STK19 | ENSG00000204344 |
| STK40 | ENSG00000196182 |
| SUFU | ENSG00000107882 |
| SUZ12 | ENSG00000178691 |
| SYK | ENSG00000165025 |
| TAP1 | ENSG00000168394 |
| TAP2 | ENSG00000204267 |
| TBX3 | ENSG00000135111 |
| TCF3 | ENSG00000071564 |
| TCF7L2 | ENSG00000148737 |
| TEK | ENSG00000120156 |
| TENT5C | ENSG00000183508 |
| TERT | ENSG00000164362 |
| TET1 | ENSG00000138336 |
| TET2 | ENSG00000168769 |
| TGFBR1 | ENSG00000106799 |
| TGFBR2 | ENSG00000163513 |
| TMEM127 | ENSG00000135956 |
| TMPRSS2 | ENSG00000184012 |
| TNFAIP3 | ENSG00000118503 |
| INFRSF14 | ENSG00000157873 |
| TOP1 | ENSG00000198900 |
| TP53 | ENSG00000141510 |
| TP53BP1 | ENSG00000067369 |
| TP63 | ENSG00000073282 |
| TRAF2 | ENSG00000127191 |
| TRAF7 | ENSG00000131653 |
| TSC1 | ENSG00000165699 |
| TSC2 | ENSG00000103197 |
| TSHR | ENSG00000165409 |
| U2AF1 | ENSG00000160201 |
| UPF1 | ENSG00000005007 |
| VEGFA | ENSG00000112715 |
| VHL | ENSG00000134086 |
| VTCN1 | ENSG00000134258 |
| WT1 | ENSG00000184937 |
| WWTR1 | ENSG00000018408 |
| XIAP | ENSG00000101966 |
| XPO1 | ENSG00000082898 |
| XRCC2 | ENSG00000196584 |
| YAP1 | ENSG00000137693 |
| YES1 | ENSG00000176105 |
| ZFHX3 | ENSG00000140836 |
| ZRSR2 | ENSG00000169249 |

Supplementary Table 4

[symbol]

| Name | Sequence (5′→3′) | [symbol] | Note |
|---|---|---|---|
| Reverse | TTCTAATACGACTCACTATAGGGCTCTTCG | 25 nmol, Desalting | Recognition sequence of Nt.BspQI: |
| complement oligo | | | 5′-GCTCTTON-3′ |

[symbol]

| Name | Sequence (5′→3′) | [symbol] | Note |
|---|---|---|---|
| Oligo-dT30VN | AAGCAGTGGTATCAACGCAGAGTACTTTTTTTTTTTTTT | 100 nmol, HPLC | Reverse transcription of poly(A)+ RNA |
| | TTTTTTTTTTTTTTTTV | | |
| TSO | AAGCAGTGGTATCAACGCAGAGTACATTGTGTG | 250 nmol, RNase-free HPLC | Template switching reaction |
| [symbol] | AAGCAGTGGTATCAACGCAGAGT | 25 nmol, Desalting | PCR |

[symbol]

| Name | Sequence (5′→3′) | [symbol] | Note |
|---|---|---|---|
| Oligo-dT30VN | ACTTGCCTGTCGCTCTATCTTCTTTTTTTTTTTTTTTTT | 100 nmol, HPLC | Reverse transcription of poly(A)+ RNA |
| | TTTTTTTTTTTTTVN | | |
| TSO | TTTCTGTTGGTGCTGATATTGCTTTIGIGIG | 250 nmol, RNase-free HPLC | Template switching reaction |
| PCR forward primer | TTTCTGTTGGTGCTGATATTGC | 25 nmol, Desalting | PCR |
| PCR forward primer | ACTTGCCTGTCGCTCTATO | 25 nmol, Desalting | PCR |

[symbol]

| Name | Sequence (5′→3′) | [symbol] | Note |
|---|---|---|---|
| TP53_1 | GTGTGGTGGTGCCCTATGAG | 25 nmol, Desalting | Forward primer, anneal to exon 6 of TP53, used |
| | | | for RT-PCR and Sanger sequencing of cDNA |
| TP53_2 | ATGATGGTGAGGATGGGCCT | 25 nmol, Desalting | Revese primer, anneal to exon 7 of TP53, used |
| | | | for both RT-PCR and Sanger sequencing |
| TP53_3 | AGCTTACAGAGGCTAAGGGG | 25 nmol, Desalting | Forward primer anneal to intron 6 of TP53, used |
| | | | for Sanger sequencing of gDNA |
| Notch1_1 | AGCCGGGGAAGAGAGGG | 25 nmol, Desalting | Forward primer, anneal to exon 1 of NOTCH1, |
| | | | used for RT-PCR |
| Notch1_2 | CTTCTTGCTGGCCTCAGACA | 25 nmol, Desalting | Reverse primer, anneal to exon 28 of NOTCH1, |
| | | | used for RT-PCR |
| Notch1_3 | CTTCAAGCAGGACGTGTTCC | 25 nmol, Desalting | Forward primer, anneal to intron 1 of NOTCH1, |
| | | | used for Sanger sequencing of gDNA |
| Notch1_4 | CCCACGAAGAACAGAAGCACA | 25 nmol, Desalting | Reverse primer, anneal to exon 28 of NOTCH1, |
| | | | used for Sanger sequencing of gDNA |
| RB1_105_1 | AGGATCTTCCTCATGCTGTTCA | 25 nmol, Desalting | Forward primer, anneal to exon 21 of RB1, |
| | | | used for RT-PCR |
| RB1_105_2 | GTTGGTGTTCGCAGACCTTC | 25 nmol, Desalting | Reverse primer, anneal to exon 23 of RB1, |
| | | | used for RT-PCR |
| RB1_105_3 | AGAAGAGCAGCTATAATCCAAGG | 25 nmol, Desalting | Forward primer, anneal to intron 21 of RB1, |
| | | | used for Sanger sequencing of gDNA |
| RB1_105_4 | CCTCCAGGAATCCGTAAGGG | 25 nmol, Desalting | Reverse primer, anneal to exon 23 of RB1, |
| | | | used for Sanger sequencing of gDNA |

[symbol]

indicates data missing or illegible when filed

### Example 6—Materials and Methods

Cell lines. SH-SY5Y human neuroblastoma cells (ATCC, #CRL-2266) were cultured in DMEM/F-12 (Gibco, #11330032) supplemented with 10% fetal bovine serum (FBS, Corning, #45000-734) and 100 U/ml penicillin-streptomycin (Gibco, #15140122). SH-SY5Y cells were maintained at 37° C. in a humidified chamber with 5% CO₂. The SH-SY5Y cell line was authenticated by short tandem repeat analysis and verified to be mycoplasma-free. A panel of 40 breast cancer cell lines was obtained from the American Type Culture Collection (ATCC, Manassas, VA, USA 30-4500 K™). Cell lines were cultured according to ATCC recommendations and were authenticated by the supplier.

RNA extraction and preparation. Spike-in RNA variants (SIRV-Set 4, Lexogen, #141.01) were aliquoted immediately upon arrival (5 ng per tube). One aliquot of SIRVs was further diluted by 1:1000 to 5 pg/μl as a working concentration for reverse transcription. Human brain total RNA (50 μg, Clontech, Cat. #636530, Lot. #2006022) was isolated from pooled tissues of multiple donors, as indicated by the manufacturer. Total RNA was extracted from the SH-SY5Y cell line and 40 breast cancer cell lines using TRIzol reagent (Invitrogen, #15596018). RNA concentrations and RNA integrity were measured with a NanoDrop 2000 Spectrophotometer and Agilent 4200 TapeStation, respectively.

RT-PCR validation and Sanger sequencing of cDNA. Total RNA was treated with RNase-free DNase I by using the TURBO DNA-free Kit (Invitrogen, Cat. AM1907). The cDNA was synthesized from 1 μg of total RNA by using oligo (dT)15 primed reverse transcription, by following the Maxima H minus reverse transcriptase protocol. Next, PCR was performed in a 20-ul volume by using first-strand cDNA synthesized from 50 ng of total RNA, 10 μl of KAPA HiFi ReadyMix, and 10 pmol of a primer pair. All primer pairs are listed in Supplementary Table 4. PCR amplification was carried out in a Veriti 96-well Thermal Cycler (Applied Biosystems, Cat. #43-757-86) by incubating the mixture at 95° C. for 3 min, followed by 26 cycles of (98° C. for 20 s, 65° C. for 20 s, and 72° C. for 45 s) with a final extension at 72° C. for 2 min. Amplified products were analyzed by electrophoresis in 2% agarose gels and a D1000 ScreenTape assay on an Agilent 4200 TapeStation. Splice junction sequences of transcript isoforms were confirmed by Sanger sequencing of the DNA amplicon, which were separated by DNA electrophoresis. Gel extraction was performed using the QIAquick Gel Extraction Kit (Qiagen, Cat. #28706X4).

Genomic DNA isolation and Sanger sequencing validation. Genomic DNA was isolated using TRIzol reagent (Invitrogen) according to the DNA isolation protocol from TRIzol. DNA concentration and integrity were measured by a NanoDrop 2000 Spectrophotometer and Genomic DNA ScreenTape assay on an Agilent 4200 TapeStation, respectively. PCR was performed in a 50-μl volume using 50 ng of genomic DNA, 25 μl of KAPA HiFi ReadyMix, and 20 pmol of a primer pair. All primer pairs are listed in Supplementary Table 4. PCR amplification was carried out in a Veriti 96-well Thermal Cycler (Applied Biosystems, Cat. #43-757-86) by incubating the mixture at 95° C. for 3 min, followed by 30 cycles of (98° C. for 20 s, 65° C. for 20 s, and 72° C. for 1 min) with a final extension at 72° C. for 2 min. Amplified products were separated by electrophoresis in 1.5% agarose gels, and bands were purified with QIAquick Gel Extraction Kit (Qiagen, Cat. #28706X4). Sequences of purified DNA amplicons were confirmed using Sanger sequencing with the same primer used in PCR.

Short-read RNA-seq library preparation and sequencing. Short-read sequencing libraries were prepared with 1 μg of total RNA extracted from SH-SY5Y cells, together with 25 μg of SIRV-set4 RNA, following the TruSeq Stranded mRNA protocol (Illumina, Cat. #20020595). All short-read libraries (n=3) were sequenced on an Illumina NovaSeq 6000 sequencer with 150-bp paired-end sequencing, according to the manufacturer's protocol.

Direct RNA library construction and nanopore sequencing. A 20-μg aliquot of total RNA was subjected to poly(A)+ RNA selection using the Dynabeads mRNA DIRECT purification kit (Invitrogen, #61011) following the manufacturer's instructions. Approximately 500 ng of the resulting poly(A)+ RNA, along with 5 ng of SIRVs, were pooled as input for direct RNA library generation. Libraries were made by following the standard ONT SQK-RNA002 protocol with the optional reverse transcription step included. All libraries were loaded onto R9.4.1 flow cells and sequenced on MinION/GridION devices (ONT, Oxford, UK).

Full-length cDNA synthesis. A 200-ng aliquot of total RNA, together with 5 μg of SIRV-Set 4 RNA, were used as templates for cDNA synthesis. Briefly, the reverse transcription and template-switching reaction was performed by using Maxima H minus reverse transcriptase (Thermo Scientific, #EP0751) under the following conditions: 42° C. for 90 min, followed by 85° C. for 5 min. First-strand cDNA was amplified by PCR with KAPA HiFi ReadyMix (KAPA Biosystems, #KK2602) by incubating the mixture at 95° C. for 3 min, followed by 11 cycles of (98° C. for 20 s, 67° C. for 20 s, and 72° C. for 5 min) with a final extension at 72° C. for 8 min. PCR products were purified using 0.8× volumes of SPRIselect beads (Beckman Coulter, #B23318). Amplified cDNA was measured using the Qubit dsDNA High Sensitivity assay and Agilent High Sensitivity D5000 ScreenTape assay on a 4200 TapeStation. Sequences of oligos/primers are detailed in Supplementary Table 4.

1D library construction and nanopore sequencing. Nanopore 1D libraries were constructed using 1 μg of amplified cDNA according to the standard ONT SQK-LSK109 protocol. Briefly, cDNA products were end-repaired and dA-tailed using NEBNext Ultra II End Repair/dA-Tailing Module (NEB, #E7546) by incubating at 20° C. for 20 min and 65° C. for 20 min. The cDNA was then purified with 1× volume of AMPure XP beads and eluted in 60 μl of nuclease-free water. Adapter ligation was performed using NEBNext Quick T4 DNA ligase (NEB, #E6056) at room temperature for 10 min. After ligation, libraries were purified using 0.45× volumes of AMPure XP beads and short fragment buffer. The final libraries were loaded onto R9.4.1 flow cells and sequenced on MinION/GridION devices.

Capture probe synthesis. IDT Lockdown probes (Integrated DNA Technologies) were designed and synthesized for a test panel of 10 brain genes, including HTT, MAPT, RBFOX1, NRXN1, NUMB, DAB1, GRIN1, SCN8A, DLG4, and LRP8. The probes are 120-nt long oligos that are biotinylated at their 5′ ends. Probes were designed to tile across all annotated exons, including UTRs, of test panel genes with 1× tiling density (Supplementary Table 4).

TEQUILA probes were synthesized in two steps. First, Twist oligo pools (Twist Bioscience) were designed and synthesized for 3 custom-designed gene panels, which are detailed in Supplementary Table 4. The oligos are 150-nt long and contain a 30-nt universal primer binding sequence (5′-CGAAGAGCCCTATAGTGAGTCGTATTAGAA-3′) at the 3′ end. The remaining 120 nt are designed to tile across all annotated exons, including UTRs, of targeted genes with 1× tiling density. Next, oligo pools were amplified and biotin-labeled using nickase-induced linear SDA. Briefly, a 40 μl of reaction volume containing 2-10 ng of the oligo pool as ssDNA templates, 5 μl of 10×NEBuffer 3.1, 2 mM DTT, 0.25 μM RC-oligo (5′-TTCTAATACGACTCACTATAGGGCTCTTCG-3′), 0.4 mM dTTP, 0.6 mM dATP, 0.6 mM dCTP, 0.6 mM dGTP, and 0.2 mM biotin-dUTP was assembled on ice. The mixture was incubated at 95° C. for 2 min, and then ramped down to 4° C. at a rate of 0.1° C./s. Initial strand extension of primers was performed at 37° C. for 10 min using 5 μM of ssDNA binding protein (T4 Gene 32 Protein, NEB, Cat. #M0300S) and 0.8 U/ul of Klenow Fragment (3′-5′ exo-) DNA polymerase (NEB, Cat. #M0212M). Nickase-induced linear SDA was then performed at 37° C. for 12-16 h using 3 nM (0.04 U/ul) of Nt.BspQI (NEB, Cat. #R0644S). Synthesized probes were purified with 1.8× volumes of AMPure XP beads and quantified by NanoDrop 2000 Spectrophotometer.

Hybridization and capture. All hybridization and capture experiments were done following a protocol from IDT (“Hybridization capture of DNA libraries using xGen Lockdown probes and reagents”). Briefly, approximately 500 ng of amplified cDNA were denatured at 95° C. for 10 min and then incubated with either 3 pmol of IDT xGen Lockdown probes or 100 ng of TEQUILA probes at 65° C. for 12 h. Next, 50 μl of M-270 streptavidin beads (Invitrogen, Cat. #65306) were added to the mixture, which was incubated at 65° C. for 45 min. The mixture was then immediately subjected to a series of high-temperature and room temperature washes, according to the IDT xGen Lockdown protocol. The resulting bead solution was resuspended in 40 μl of TE buffer.

Post-capture amplification and nanopore sequencing. On-bead PCR was performed for the streptavidin bead-captured cDNA using KAPA HiFi ReadyMix by incubating at 95° C. for 3 min, followed by 12 cycles of (98° C. for 20 s, 67° C. for 20 s, 72° C. for 5 min), with a final extension at 72° C. for 8 min. PCR products were purified using 0.7× volumes of SPRIselect beads. Amplified cDNA was subjected to 1D library construction and nanopore sequencing.

Basecalling and alignment of nanopore sequencing data. Basecalling of raw nanopore data was performed in fast mode using Guppy (v4.0.15) with the following settings: ‘guppy_basecaller -input_path raw_data -save_path output_folder -config corresponding_config_file’ (community.nanoporetech.com/downloads). Basecalling of 1D cDNA sequencing and TEQUILA-seq data was done using config file ‘dna_r9.4.1_450bps_fast.cfg’, and basecalling of direct RNA sequencing data was done using config file ‘rna_r9.4.1_70bps_fast.cfg’.

Basecalled reads were mapped to either the GRCh37/hg19 reference genome or SIRV genome from Lexogen (SIRV-Set 4) using minimap2 (v2.17) with parameters: ‘-a -x splice -ub -k 14 -w 4 -secondary=no’. Specifically, the inventors provided minimap2 transcript annotations from GENCODE v34 (world-wide-web at gencodegenes.org/human/release_34lift37.html) when mapping reads to the GRCh37/hg19 reference genome. They provided SIRV-Set 4 transcript annotations when mapping reads to the SIRV genome.

Discovery and quantification of transcript isoforms. Full-length transcript isoforms were detected and quantified from long-read alignment files using ESPRESSO (v1.2.2) with default settings (github.com/Xinglab/espresso). Specifically, ESPRESSO was used to simultaneously identify and quantify transcript isoforms from the following sets of nanopore RNA-seq data:
1. 1D cDNA sequencing data and targeted sequencing data (IDT probes or TEQUILA probes) of 10 test genes on human brain cDNA samples (n=3 per sequencing protocol).
2. Direct RNA sequencing data, 1D cDNA sequencing data, and TEQUILA-seq data (4, 8, and 48 h of sequencing time) of a panel of 54 total SIRV, long SIRV, and ERCC genes on SH-SY5Y cells (n=3 per sequencing protocol).
3. Direct RNA sequencing data, 1D cDNA sequencing data, and TEQUILA-seq data (4, 8, and 48 h of sequencing time) of a panel of 221 genes encoding splicing factors on SH-SY5Y cells (n=3 per sequencing protocol).
4. TREQUILA-seq data of 468 actionable cancer genes (Supplementary Table 3) on 40 breast cancer cell lines (n=2 per cell line).
5. 1D cDNA sequencing data on 4 breast cancer cell lines: HCC1806, MDA-MB-157, AU-565, and MCF7 (n=1 per cell line).

Estimated read counts for all transcript isoforms identified in a sample (i.e., those with a nonzero read count) were normalized into counts per million (CPM) by dividing the number of reads assigned to a transcript isoform by the total number of reads mapped to the reference genome and multiplying this number by one million. The proportion of a transcript isoform was calculated by dividing the CPM value of a transcript by the CPM value of the corresponding gene (i.e., sum of CPM values over all transcripts discovered for the gene).

Calculation of on-target rate and fold enrichment. For each sample subjected to targeted sequencing, the inventors computed an on-target rate by dividing the number of reads mapped to targeted genes (with mapping quality score ≥1) by the total number of reads aligned to the reference genome (with mapping quality score ≥1). To characterize the overall on-target rate for a given targeted enrichment method, the inventors calculated the mean and standard deviation of on-target rates across all replicates associated with the method. Fold enrichment was calculated by dividing the mean on-target rate for a targeted enrichment method by the mean on-target rate across non-capture control samples.

Quantification of exon skipping events using short- and long-read RNA-seq data. The inventors aligned short-read RNA-seq data to the GRCh37/hg19 reference genome using STAR (v2.6.1d) on two-pass mode with default settings and transcript annotations from GENCODE v34 (world-wide-web at gencodegenes.org/human/release_34lift37.html). Exon skipping events were detected and quantified (as percent spliced in, ψ) from short-read alignment files using rMATS (v4.1.1) with default settings (Shen et al., 2014).

For each exon skipping event identified from short-read data, the inventors also computed ψ values based on long-read data using the following equation:

$$ψ = \frac{I}{I + S}$$

where I is the sum of CPM values for transcripts carrying both of the inclusion junctions associated with the exon skipping event, and S is the sum of CPM values for transcripts carrying only the skipping junction associated with the exon skipping event.

Detection of high-confidence exon skipping events from short-read RNA-seq data. The inventors identified high-confidence exon skipping events from short-read RNA-seq data based on the following criteria: (1) the average number of short reads spanning both exon-inclusion junctions or the number of short reads supporting the exon skipping junction is ≥10, (2) the ratio between the average number of short reads supporting either exon-inclusion junction is between 0.2 and 5, (3) the average short-read y value is between 0.01 and 0.99, and (4) none of the 4 splice sites associated with the exon skipping event is involved in other AS events detected from short-read RNA-seq data.

Identification of breast cancer subtype-specific transcript isoforms. The inventors sought to identify transcript isoforms that are breast cancer subtype-specific using a panel of 40 breast cancer cell lines. For each breast cancer subtype (luminal, HER2-enriched, basal A, or basal B), the inventors used a two-sided Student's t-test to compare the mean proportion of a transcript isoform between cell lines associated with the given subtype and all other cell lines. They subsequently identified tumor subtype-specific transcript isoforms as those satisfying the following criteria: (1) FDR-adjusted p-value≤5% based on Benjamini-Hochberg correction, and (2) the mean isoform proportion across cell lines of the given subtype is greater than the mean isoform proportion over all other cell lines by at least 10%.

Identification of tumor-aberrant transcript isoforms. The inventors defined “tumor-aberrant transcript isoforms” as transcript isoforms with increased usage in at least 1 but no more than 4 cell lines in the panel of 40 breast cancer cell lines (≤10% of cell lines). To identify such transcript isoforms, the inventors used the following statistical procedure:

For each gene, the inventors generated an m-by-80 contingency table comprised of read counts (rounded to the nearest integer) for m discovered transcript isoforms across 80 TEQUILA-seq samples (2 technical replicates for each of the 40 breast cancer cell lines). Using this matrix, the inventors computed total gene expression levels in each sample as the sum of read counts over all transcript isoforms of the gene. They ignored genes that only had one identified isoform or were only expressed in a single sample. They also omitted samples from the contingency table if the given gene was not expressed in those samples.

Next, the inventors ran a chi-square test of homogeneity (FDR<1%) on the matrix to assess whether transcript isoform proportions for the given gene are homogenous across the considered samples. Focusing on genes prioritized by the chi-square test with FDR<1%, the inventors ran a post-hoc test to identify sample-isoform pairs in which the isoform proportion in the given sample is significantly higher than the overall isoform proportion across all samples (i.e., sum of read counts of the transcript isoform over all samples divided by the sum of read counts of the gene over all samples) (one-tailed binomial test, FDR<1%).

Using transcript isoforms prioritized by this post-hoc test, the inventors next identified cell line-isoform pairs for which the transcript isoform shows significantly elevated usage in a given cell line (i.e., known as “cell-line enriched” isoforms). Specifically, these pairs were required to satisfy the following criteria: (1) the transcript isoform has an adjusted p-value <1% (post-hoc test) using the Benjamini-Hochberg correction for both replicate samples associated with the given cell line, and (2) the transcript isoform proportions in both replicate samples are ≥10% higher than the transcript isoform proportion over all samples.

Finally, the inventors defined a set of tumor-aberrant transcript isoforms based on the following requirements: (1) the transcript isoform shows significantly elevated usage in at least 1 but no more than 4 cell lines (i.e., ≤10% of the inventors' breast cancer cell line panel), and (2) the transcript isoform is not the canonical transcript isoform of the corresponding gene. Canonical transcript isoforms for each gene were identified using the Ensembl database (Release 100, April 2020). A custom script for identifying tumor-aberrant transcript isoforms is available at [insert GitHub link].

Classification of AS events underlying tumor-aberrant transcript isoforms. To characterize RNA processing changes associated with tumor-aberrant transcript isoforms, the inventors directly compared the structure of each tumor-aberrant transcript isoform with the structure of the canonical transcript isoform for the corresponding gene. Local differences in transcript structure were classified into 7 basic AS categories (Park et al., 2018), including: (1) exon skipping, (2) alternative 5′-splice site, (3) alternative 3′-splice site, (4) mutually exclusive exons, (5) intron retention, (6) alternative first exon, and (7) alternative last exon. Any local differences in transcript structure that could not be classified as one of the 7 basic categories were classified as “complex splicing”. If a tumor-aberrant transcript isoform was found to have more than one AS event relative to the canonical transcript isoform, it was labeled as “combinatorial”. In comparisons of transcript structure, the inventors filtered out tumor-aberrant transcript isoforms that (i) were also the canonical transcript isoform of the corresponding gene, or (ii) only differed in transcript ends relative to the canonical transcript isoform. They wrote a custom script (available at github.com/Xinglab/TEQUILA-seq that identifies structural differences between two transcript isoforms and classifies these differences into different AS categories.

Identification of NMD-targeted transcripts. All transcript isoforms identified by ESPRESSO were classified into the following 3 categories: (1) transcripts annotated in GENCODE (v34lift37) as ‘basic’ (i.e., full-length) protein-coding or targeted by NMD, (2) transcripts annotated in GENCODE but not labeled as ‘basic’ protein-coding or targeted by NMD, (3) novel transcripts identified by ESPRESSO. For transcripts assigned to category (2) or (3), the inventors retrieved their sequences relative to the GRCh37/hg19 reference genome and searched for ORFs. Specifically, they used the longest ORF for a given transcript and required it to encode at least 20 amino acids.

Among transcripts with predicted ORFs, the inventors identified those that may be targeted by NMD using the following criteria: (1) the transcript is ≥200 nt long, (2) the transcript contains at least one splice junction, and (3) the predicted stop codon is ≥50 nt upstream of the last exon-exon junction (i.e., the transcript harbors a PTC) (Kurosaki et al., 2019).

Enrichment analysis of NMD-targeted tumor-aberrant transcript isoforms for tumor-suppressor genes (TSGs) and oncogenes (OGs). The inventors categorized the 468 actionable cancer genes as either TSGs or OGs based on annotations from OncoKB (world-wide-web at oncokb.org) (Chakravarty et al., 2017). Among the 468 genes, 196 were annotated as TSGs, 179 were annotated as OGs, and the remaining 93 genes were assigned to “Other” category, referring to genes with context-dependent behavior as either a TSG or an OG as well as genes with unknown functions in the context of cancer.

The inventors sought to examine whether NMD-targeted tumor-aberrant isoforms are enriched in TSGs compared to OGs. First, they filtered their list of 468 actionable cancer genes for those that were detected (average gene CPM of two replicates ≥1) in at least 10 of the 40 breast cancer cell lines. From this list of expressed genes, the inventors next counted the number of TSGs and OGs with or without NMD-targeted tumor-aberrant transcript isoforms and organized the count data into a 2×2 contingency table. Finally, the inventors used a Fisher's exact test on this contingency table to evaluate whether having NMD-targeted tumor-aberrant isoforms is associated with TSGs. Moreover, for each cell line, they calculated the proportion of expressed TSGs, OGs, and “Other” genes that also express NMD-targeted tumor-aberrant transcript isoforms in that cell line (average gene CPM of 2 replicates ≥1). The inventors used a two-sided paired Wilcoxon test to assess whether the distributions of these proportion values across all 40 breast cancer cell lines differed between TSGs and OGs.

### III. References

The following references, to the extent that they provide exemplary procedural or other details supplementary to those set forth herein, are specifically incorporated herein by reference.
Amarasinghe et al., Genome Biol 21, 30 (2020).
Baralle & Giudice, Nat Rev Mol Cell Biol 18, 437-451 (2017).
Beaubier et al., Nat Biotechnol 37, 1351-1360 (2019).
Bianchini et al., Nat Rev Clin Oncol 19, 91-113 (2022).
Blencowe, Cell 126, 37-47 (2006).
Bolisetty et al., Genome Biol 16, 204 (2015).
Braunschweig et al., Cell 152, 1252-69 (2013).
Bonnal et al., Nat Rev Clin Oncol 17, 457-474 (2020).
Broseus & Ritchie, Comput Struct Biotechnol J 18, 501-508 (2020).
Byrne et al., Philos Trans R Soc Lond B Biol Sci 374, 20190097 (2019).
Byrne et al., Nat Commun 8,16027 (2017).
Chakravarty & Solit, Nat Rev Genet 22, 483-501 (2021).
Chakravarty et al., JCO Precis Oncol 2017 (2017).
Cheng et al., J Mol Diagn 17, 251-264 (2015).
Clark et al., Mol Psychiatry 25, 37-47 (2020).
Cummings et al., Sci Transl Med 9 (2017).
Dai et al., J Cancer 8, 3131-3141 (2017).
Deng et al., Nat Biotechnol 27, 353-360 (2009).
Dvinge et al., Nat Rev Cancer 16, 413-430 (2016).
Ellis et al., Mol Cell 46, 884-92 (2012).
Feng et al., Proc Natl Acad Sci USA 118, (2021).
Fiala et al., Nat Cancer 2, 357-365 (2021).
Gabrieli et al., Nucleic Acids Res 46, e87 (2018).
Garber et al., Nat Methods 8, 469-77 (2011).
Ghandi et al., Nature 569, 503-508 (2019).
Gilpatrick et al., Nat Biotechnol 38, 433-438 (2020).
Hafner et al., Nat Rev Mol Cell Biol 20, 199-210 (2019).
Han et al., Nature 498, 241-245 (2013).
Harbeck et al., Nat Rev Dis Primers 5, 66 (2019).
Heyer et al., Nat Commun 10, 1388 (2019).
Horak et al., Cancer Discov 11, 2780-2795 (2021).
Hughes et al., Nat Genet 46, 205-212 (2014).
Jiang et al., Genome Res 21, 1543-1551 (2011).
Joglekar et al., Nat Commun 12, 463 (2021).
Kalsotra & Cooper, Nat Rev Genet 12, 715-29 (2011).
Karamitros & Magiorkinis, Methods Mol Biol 1712, 43-51 (2018).
Kastenhuber & Lowe, Cell 170, 1062-1078 (2017).
Kovaka et al., Nat Biotechnol 39, 431-441 (2021).
Kozarewa et al., Curr Protoc Mol Biol 112, 7 21 1-7 21 23 (2015).
Kurosaki et al., Nat Rev Mol Cell Biol 20, 406-420 (2019).
Lagarde et al., Nat Genet 49, 1731-1740 (2017).
Lareau et al., Nature 446, 926-929 (2007).
Leclair et al., Mol Cell 80, 648-665 e649 (2020).
Lehmann et al., J Clin Invest 121, 2750-2767 (2011).
Liu et al., Genome Biol 21, 54 (2020).
Long et al., Biochem J 417, 15-27 (2009).
Loose et al., Nat Methods 13, 751-4 (2016).
Mamanova et al., Nat Methods 7, 111-118 (2010).
McCord et al., Mol Cell 77, 688-708 (2020).
Mercer et al., Nat Protoc 9, 989-1009 (2014).
Neve et al., Cancer Cell 10, 515-527 (2006).
Nilsen et al., Nature 463, 457-463 (2010).
Okano et al., Cell 99, 247-257 (1999).
Pan et al., Nat Genet 40, 1413-1415 (2008).
Pan et al., Trends Pharmacol Sci 42, 268-282 (2021).
Park et al., Am J Hum Genet 102, 11-26 (2018).
Paronetto et al., Cell Death Differ 23, 1919-1929 (2016).
Paul et al., bioRxiv, 080747 (2016).
Payne et al., Nat Biotechnol, 2021. 39 (4): p. 442-450.
Reeser et al., J Mol Diagn 19, 682-696 (2017).
Rhee et al., Nature 416, 552-556 (2002).
Sahlin et al., Nat Commun 9, 4601 (2018).
Sathasivam et al., Proc Natl Acad Sci USA 110, 2366-2370 (2013).
Scotti & Swanson, Nat Rev Genet 17, 19-32 (2016).
Shalek et al., Nature 498, 236-40 (2013).
Shen et al., Proc Natl Acad Sci USA 111, E5593-5601 (2014).
Sheynkman et al., Nat Commun, 2020. 11 (1): p. 2326
Shukla et al., Nat Commun 13, 2485 (2022).
Staaf et al., Nat Med 25, 1526-1533 (2019).
Stark et al., Nat Rev Genet 20, 631-656 (2019).
Steijger et al., Nat Methods 10, 1177-84 (2013).
Sun et al., Sci Rep 8, 11646 (2018).
Tang et al., Nat Commun 11,1438 (2020).
Tardaguila et al., Genome Res, (2018).
Vaquero-Garcia et al., Elife 5, e11752 (2016).
Veiga et al., Sci Adv 8, eabg6711 (2022).
Vuong et al., Nat Rev Neurosci 17, 265-281 (2016).
Wade-Martins, Nat Rev Neurol 8, 477-478 (2012).
Wallace & Bean, Gene Reviews, 1993-2021, University of Washington, Seattle.
Wang et al., Nature 456, 470-476 (2008).
Wang et al., Nat Biotechnol 39, 1348-1365 (2021).
Wang & Rio, Proc Natl Acad Sci USA 115, E8181-E8190 (2018).
Wilson et al., Toxicol Sci 66, 69-81 (2002).
Xu et al., Nucleic Acids Res 30, 3754-66 (2002).

1. A method of preparing a panel of biotinylated oligonucleotide probes, the method comprising:
(a) obtaining a set of oligonucleotides, each comprising a target gene binding sequence at its 5′ end and a primer binding sequence at its 3′ end, wherein each oligonucleotide has the same the primer binding sequence, and wherein the 5′ end of the primer binding sequence comprises a nickase target sequence;
(b) incubating the set of oligonucleotides with a primer that hybridizes to the primer binding sequence and with biotinylated dNTP (e.g., biotin-dUTP) under conditions to allow for extension of the primer using the oligonucleotides as a template, thereby producing extended primers complementary to the oligonucleotides, where the extended primers each comprise, from 5′ to 3′, the primer, the nickase target sequence, and a biotinylated probe;
(c) nicking the extended primers complementary to the oligonucleotides with a nickase capable of cleaving the extended primers at the nickase target sequence to separate the biotinylated probes and regenerate the primers' 3′ end;
(d) extending the regenerated primers 3′ end using the oligonucleotides as templates to displace and release the biotinylated probes; and
(e) repeating steps (c) and (d).

2. The method of claim 1, wherein each oligonucleotide in the set is about 60 to 150 nucleotides long.

3. The method of claim 1, wherein each oligonucleotide in the set comprises a 30 to 120-nucleotide sequence at its 5′ end that is capable of hybridizing to a target gene and a 30-nucleotide primer binding site at its 3′ end.

4. The method of claim 3, wherein the 30-nucleotide primer binding site has one of the following sequences depending on the nickase used and selected from 1) Nt.BspQI: 5′-NGAAGAGCCCTATAGTGAGTCGTATTAGAA-3′; 2) Nt.BstNBI: 5′-NNNNGACTCCCTATAGTGAGTCGTATTAGAA-3′; 3) Nb.AlwI: 5′-NNNNGATCCCCTATAGTGAGTCGTATTAGAA-3′; and 4) Nt.BsmAI: 5′-NGAGACCCTATAGTGAGTCGTATTAGAA-3′, wherein 5′-CCTATAGTGAGTCGTATTAGAA-3′ is a universal primer sequence and the italicized bases are targeting sequences.

5. The method of claim 3, wherein within the set of oligonucleotides, the 30 to 120-nucleotide 5′ end sequences are tiled across the sequence of each target gene.

6. The method of claim 5, wherein the oligonucleotides are tiled at about or greater than a density of 0.5×, 1×, or 2× across the sequence of each target gene.

7. The method of claim 5, wherein oligonucleotides are tiled across the targeted gene sequence regions, including, but not limited to genomic DNA or RNA sequences of target genes including the exon sequences, or/and the intronic sequences.

8. The method of claim 1, wherein step (b) comprises (i) combining the set of oligonucleotides, the primer, deoxynucleotides, and biotinylated dNTP (e.g., biotin-dUTP) and incubating the mixture at 95° C. for 2 min, followed by a slow ramp-down (−0.1° C./s) to 4° C.; and (ii) adding a single-stranded DNA binding protein and a DNA polymerase that exhibits 5′ to 3′ strand displacement activity and incubating at a temperature between 20° C. and 37° C. for initial primer extension.

9. The method of claim 8, wherein the DNA polymerase that harbors 5′ to 3′ strand displacement activity includes, but not limited to Klenow Fragment (3′→5′ exo-) DNA polymerase; Hemo KlenTaq DNA polymerase; Bst DNA Polymerase, Large Fragment; Bst DNA Polymerase; Bsu DNA Polymerase, Large Fragment; phi29 DNA Polymerase; and Vent® (exo-) DNA Polymerase.

10. The method of claim 1, wherein steps (c)-(e) comprise adding a nickase to the reaction and incubating at a temperature between 20° C. and 37° C.

11. The method of claim 10, wherein the incubating occurs for between 30 min and 24 h.

12. The method of claim 1, wherein steps (d) and (e) occur without any exogenous manipulation.

13. The method of claim 1, further comprising (f) isolating and/or purifying the biotinylated probes.

14. The method of claim 1, wherein the nickase can include, but are not limited to Nt.BspQI, Nt.BstNBI, Nb.AlwI, or Nt.BsmAI.

15. The method of claim 1, wherein the extension of steps (b) and (d) is performed by a DNA polymerase that harbors 5′ to 3′ strand displacement activity including, but not limited to Klenow Fragment (3′→5′ exo-) DNA polymerase; Hemo KlenTaq DNA polymerase; Bst DNA Polymerase, Large Fragment; Bst DNA Polymerase; Bsu DNA Polymerase, Large Fragment; phi29 DNA Polymerase; and Vent (exo-) DNA Polymerase.

16. The method of claim 1, wherein the method is an isothermal reaction.

17. The method of claim 1, wherein the method is performed at a temperature between 20° C. and 37° C.

18. A panel of biotinylated oligonucleotide probes made by the method of claim 1.

19. The panel of probes of claim 18, wherein each probe comprises one or more biotin-NMP residues (e.g., biotin-UMP residues).

20. The panel of probes of claim 18, wherein each probe consists of sequences that are complementary to a target nucleic acid sequence, including, but not limited to, a gene's DNA locus, transcript isoforms or an intergenic DNA region.

21. A method of sequencing a plurality of nucleic acid molecules comprising:
(a) obtaining a sample comprising the plurality of nucleic acid molecules;
(b) hybridizing the panel of probes of any one of claims 18-20 to the plurality of nucleic acid molecules;
(c) capturing the hybridized probes using streptavidin beads;
(d) amplifying the nucleic acid molecules that were bound to the captured hybridized probes; and
(e) sequencing the amplified nucleic acid molecules.

22. The method of claim 21, wherein the sequencing comprises Sanger sequencing, sequencing-by-synthesis, including, but not limited to, Illumina NGS platform sequencing and PacBio long-read sequencing, or nanopore sequencing.

23. The method of claim 21, wherein the sequencing comprises long-read sequencing.

24. The method of claim 21, wherein the sequencing comprises short-read sequencing.

25. The method of claim 21, wherein the streptavidin beads are magnetic.

26. The method of claim 21, wherein the sample is a dsDNA library, including, but not limited to cDNA library and fragmented genomic DNA library.

27. The method of claim 26, wherein the cDNA library was produced by reverse transcription-polymerase chain reaction of an RNA sample.

28. The method of claim 26, wherein the sequencing provides a transcriptomic profile.

29. The method of claim 28, wherein the transcriptomic profile includes gene expression changes and RNA splicing changes.

30. The method of claim 21, wherein the method is a method of targeted sequencing of full-length transcripts, non-full-length transcripts or any genomic fragments.


SEQUENCE LISTING (7 sequences)

SEQ ID NO: 1 - DNA, 30 bp, synthetic construct
  source 1..30: mol_type = other DNA
ngaagagccctatagtgagtcgtattagaa

SEQ ID NO: 2 - DNA, 31 bp, synthetic construct
  source 1..31: mol_type = other DNA
nnnngactccctatagtgagtcgtattagaa

SEQ ID NO: 3 - DNA, 31 bp, synthetic construct
  source 1..31: mol_type = other DNA
nnnngatcccctatagtgagtcgtattagaa

SEQ ID NO: 4 - DNA, 28 bp, synthetic construct
  source 1..28: mol_type = other DNA
ngagaccctatagtgagtcgtattagaa

SEQ ID NO: 5 - DNA, 22 bp, synthetic construct
  source 1..22: mol_type = other DNA
cctatagtgagtcgtattagaa

SEQ ID NO: 6 - DNA, 30 bp, synthetic construct
  source 1..30: mol_type = other DNA
cgaagagccctatagtgagtcgtattagaa

SEQ ID NO: 7 - DNA, 30 bp, synthetic construct
  source 1..30: mol_type = other DNA
ttctaatacgactcactatagggctcttcg